(19) United States
(12) Patent Application Publication
(10) Pub. No.: US 2022/0011488 A1
(43) Pub. Date: Jan. 13, 2022

(54) COMPOUND, POLYMERIZABLE COMPOSITION, CURED PRODUCT, OPTICAL FILM, POLARIZING PLATE, AND IMAGE DISPLAY DEVICE

(71) Applicant: FUJIFILM Corporation, Tokyo (JP)

(72) Inventors: Aiko YAMAMOTO, Kanagawa (JP); Hiroshi INADA, Kanagawa (JP); Shunya KATOH, Kanagawa (JP); Toshikazu SUMI, Kanagawa (JP)

(73) Assignee: FUJIFILM Corporation, Tokyo (JP)

(21) Appl. No.: 17/486,125

(22) Filed: Sep. 27, 2021

Related U.S. Application Data
(63) Continuation of application No. PCT/JP2020/013211, filed on Mar. 25, 2020.

(30) Foreign Application Priority Data
Mar. 29, 2019 (JP) 2019-068662

(51) Int. Cl. G02B 5/30; C09K 19/38; C08F 222/24; C07C 69/753

(52) U.S. Cl. CPC G02B 5/3033; C09K 19/3838; C08F 2800/20; C07C 69/753; C08F 222/245

(57) ABSTRACT

Provided is a compound having a wide temperature range exhibiting liquid crystallinity and excellent precipitation suppression and solubility; and a polymerizable composition, a cured product, an optical film, a polarizing plate, and an image display device, each using the compound. The compound is represented, for example, by Formula (1) or (2).

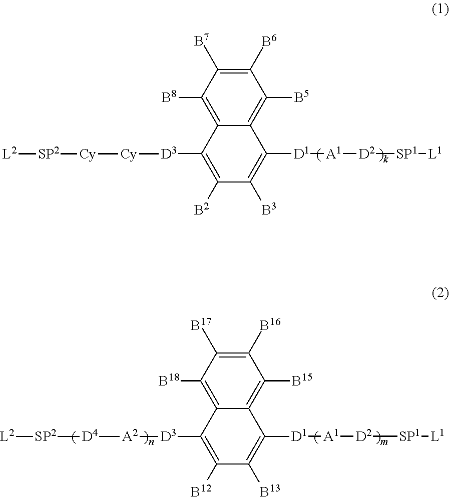

### CROSS-REFERENCE TO RELATED APPLICATIONS

This application is a Continuation of PCT International Application No. PCT/JP2020/013211 filed on Mar. 25, 2020, which claims priority under 35 U.S.C. § 119(a) to Japanese Patent Application No. 2019-068662 filed on Mar. 29, 2019. The above application is hereby expressly incorporated by reference, in its entirety, into the present application.

### BACKGROUND OF THE INVENTION

### 1. Field of the Invention

The present invention relates to a compound, a polymerizable composition, a cured product, an optical film, a polarizing plate, and an image display device.

### 2. Description of the Related Art

Optical films such as an optical compensation sheet and a phase difference film are used in various image display devices in order to eliminate image coloration or expand a viewing angle.

A stretched birefringent film has been used as the optical film, but in recent years, it has been proposed to use an optical film having an optically anisotropic layer (phase difference layer) consisting of a liquid crystalline compound instead of the stretched birefringent film.

As a polymerizable composition that forms such the optically anisotropic layer, for example, in JP2013-164520A, “a polymerizable liquid crystal composition for forming a phase difference layer, containing a first rod-shaped compound having a cyano group at one end and a (meth)acrylate at the other end, a second rod-shaped compound having (meth)acrylates at both ends, and a quaternary ammonium salt” is described ([claim 1]); and as the second rod-shaped compound, a compound represented by Formula (2) is described ([claim 3]).

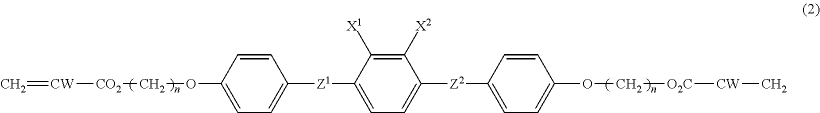

### SUMMARY OF THE INVENTION

The present inventors have conducted studies on the polymerizable composition described in JP2013-164520A, and have thus found that in a case where a ring structure is further introduced into a second rod-shaped compound in order to widen a temperature range exhibiting liquid crystallinity from the viewpoint of production characteristics and the like, the temperature range exhibiting the liquid crystallinity of the compound were wider, but the solubility of the compound was deteriorated and it was difficult to suppress the precipitation.

Therefore, an object of the present invention is to provide a compound having a wide temperature range exhibiting liquid crystallinity and excellent precipitation suppression and solubility; and a polymerizable composition, a cured product, an optical film, a polarizing plate, and an image display device, each using the compound.

The present inventors have conducted intensive studies to accomplish the object, and as a result, they have found that in a case where a compound has a predetermined structure, the compound has a wide temperature range exhibiting liquid crystallinity and excellent precipitation suppression and solubility, thereby completing the present invention.

That is, the present inventors have found that the object can be accomplished by the following configurations.

[1] A compound represented by Formula (1).

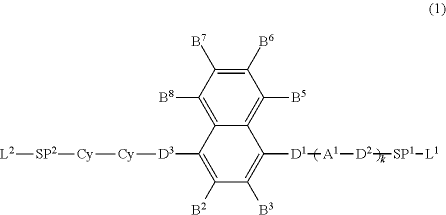

Here, in Formula (1),

A¹ represents an aromatic ring which may have a substituent or an alicyclic ring which may have a substituent.

Cy represents a 1,4-cyclohexylene group which may have a substituent, and the two Cy's may be the same as or different from each other.

D¹, D², and D³ each independently represent a single bond, or a divalent linking group consisting of —O—, —CO—, —S—, —C(═S)—, —CR¹R²—, —CR¹═CR²—, —NR¹—, or a combination of two or more thereof, and R¹ and R² each independently represent a hydrogen atom, a fluorine atom, or an alkyl group having 1 to 4 carbon atoms.

SP¹ and SP² each independently represent a single bond, a linear or branched alkylene group having 1 to 12 carbon atoms, or a divalent linking group obtained by each independently substituting one or more of —CHs-'s constituting a linear or branched alkylene group having 1 to 12 carbon atoms with —O—, —CO—, —S—, —C(═S)—, —CR¹R²—, —CR¹═CR²—, or —NR¹—, and R¹ and R² each independently represent a hydrogen atom, a fluorine atom, or an alkyl group having 1 to 4 carbon atoms.

k represents an integer of 1 to 3. In a case where k is 2 or 3, a plurality of A¹'s and D²'s which are present in the formula may be the same as or different from each other.

L¹ and L² each independently represent a monovalent organic group, and at least one of L¹ or L² represents a polymerizable group.

B², B³, B⁵, B⁶, B⁷, and B⁸ each independently represent a hydrogen atom or a substituent. It should be noted that in a case where at least one of B², B³, B⁶, or B⁷ represents a substituent, the substituent does not include a ring structure.

[2] A compound represented by Formula (2),

in which the compound is other than the compound as described in [1].

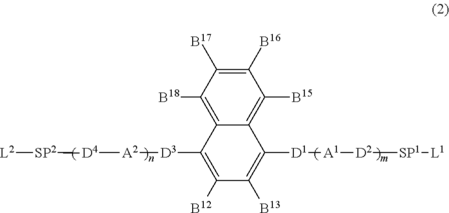

Here, in Formula (2),

A¹ and A² each independently represent an aromatic ring which may have a substituent or an alicyclic ring which may have a substituent.

D¹, D², D³, and D⁴ each independently represent a single bond, or a divalent linking group consisting of —O—, —CO—, —S—, —C(═S)—, —CR¹R²—, —CR¹—CR²—, —NR¹—, or a combination of two or more thereof, and R¹ and R² each independently represent a hydrogen atom, a fluorine atom, or an alkyl group having 1 to 4 carbon atoms.

SP¹ and SP² each independently represent a single bond, a linear or branched alkylene group having 1 to 12 carbon atoms, or a divalent linking group obtained by each independently substituting one or more of —CH₂-'s constituting a linear or branched alkylene group having 1 to 12 carbon atoms with —O—, —CO—, —S—, —C(═S)—, —CR¹R²—, —CR¹═CR²—, or —NR¹—, and R¹ and R² each independently represent a hydrogen atom, a fluorine atom, or an alkyl group having 1 to 4 carbon atoms.

m and n each independently represent an integer of 1 to 3, and satisfy m+n=an integer of 3 to 6. In a case where m is 2 or 3, A¹'s and D²'s which are present in the formula may be the same as or different from each other. In a case where n is 2 or 3, A²'s and D⁴'s which are present in the formula may be the same as or different from each other.

L¹ and L² each independently represent a monovalent organic group, and at least one of L¹ or L² represents a polymerizable group.

B¹², B¹³, B¹⁵, B¹⁶, B¹⁷, and B¹⁸ each independently represent a hydrogen atom or a substituent. It should be noted that in a case where at least one of B¹², B¹³, B¹⁶, or B¹⁷ represents a substituent, the substituent does not include a ring structure, and in a case where at least one of B¹² or B¹³ represents a substituent, the substituent does not include —CHO.

[3] The compound as described in [1],

in which at least one of B², B³, B⁵, B⁶, B⁷, or B⁸ in Formula (1) represents a substituent.

[4] The compound as described in [2],

in which at least one of B¹², B¹³, B¹⁵, B¹⁶, B¹⁷, or B¹⁵ in Formula (2) represents a substituent.

[5] The compound as described in [1] or [3],

in which at least one of B⁵ or B⁸ in Formula (1) represents a substituent.

[6] The compound as described in [2] or [4],

in which at least one of B¹⁵ or B¹⁸ in Formula (2) represents a substituent.

[7] The compound as described in [5],

in which at least one of B², B³, B⁶, or B⁷ in Formula (1) represents a hydrogen atom.

[8] The compound as described in [6],

in which at least one of B¹², B¹³, B¹⁶, or B¹⁷ in Formula (2) represents a hydrogen atom.

[9] The compound as described in [1],

in which at least one of B², B³, B⁶, or B⁷ in Formula (1) represents a substituent.

[10] The compound as described in [2],

in which at least one of B¹², B¹³, B¹⁶, or B¹⁷ in Formula (2) represents a substituent.

[11] The compound as described in [1] or [9],

in which at least one of B² or B³ in Formula (1) represents a substituent.

[12] The compound as described in [2] or [10],

in which at least one of B² or B³ in Formula (2) represents a substituent.

[13] The compound as described in [11],

in which at least one of B⁵, B⁶, B⁷, or B⁸ in Formula (1) represents a hydrogen atom.

[14] The compound as described in [12],

in which at least one of B¹⁵, B¹⁶, B¹⁷, or B¹⁸ in Formula (2) represents a hydrogen atom.

[15] The compound as described in [1] or [9],

in which at least one of B⁶ or B⁷ in Formula (1) represents a substituent.

[16] The compound as described in [2] or [10],

in which at least one of B¹⁶ or B¹⁷ in Formula (2) represents a substituent.

[17] The compound as described in [15],

in which at least one of B², B³, B⁵, or B⁸ in Formula (1) represents a hydrogen atom.

[18] The compound as described in [16],

in which at least one of B¹², B¹³, B¹⁵, or B¹⁸ in Formula (2) represents a hydrogen atom.

[19] The compound as described in [1],

in which at least one of B², B³, B⁵, B⁶, B⁷, or B⁸ in Formula (1) represents a substituent, and the substituent represents an alkyl group, an alkoxy group, an alkylcarbonyl group, an alkoxycarbonyl group, an alkylcarbonyloxy group, an alkylamino group, a dialkylamino group, an alkylamide group, an alkenyl group, an alkynyl group, a halogen group, a cyano group, a nitro group, an alkylthiol group, an N-alkylcarbamate group, an aryl group, an aryloxy group, an arylcarbonyl group, an arylcarbonyloxy group, an arylamino group, an arylamide group, an arylthiol group, an N-arylcarbamate group, a cycloalkyl group, a cycloalkyloxy group, a cycloalkylcarbonyl group, a cycloalkylcarbonyloxy group, a cycloalkylamino group, a cycloalkylamide group, a cycloalkylthiol group, an N-cycloalkylcarbamate group, a sulfonic acid ester group, or a monovalent organic group obtained by each independently substituting one or more of —CH₂-'s constituting an alkyl group with —O— or —CO—.

[20] The compound as described in [2],

in which at least one of B¹², B¹³, B¹⁵, B¹⁶, B¹⁷, or B¹⁸ in Formula (2) represents a substituent, and the substituent represents an alkyl group, an alkoxy group, an alkylcarbonyl group, an alkoxycarbonyl group, an alkylcarbonyloxy group, an alkylamino group, a dialkylamino group, an alkylamide group, an alkenyl group, an alkynyl group, halogen, a cyano group, a nitro group, an alkylthiol group, an N-alkylcarbamate group, an aryl group, an aryloxy group, an arylcarbonyl group, an arylcarbonyloxy group, an arylamino group, an arylamide group, an arylthiol group, an N-arylcarbamate group, a cycloalkyl group, a cycloalkyloxy group, a cycloalkylcarbonyl group, a cycloalkylcarbonyloxy group, a cycloalkylamino group, a cycloalkylamide group, a cycloalkylthiol group, an N-cycloalkylcarbamate group, a sulfonic acid ester group, or a monovalent organic group obtained by each independently substituting one or more of —CH₂-'s constituting an alkyl group with —O— or —CO—.

[21] A polymerizable composition comprising the compound as described in any one of [1] to [20].

[22] The polymerizable composition as described in [21], further comprising a polymerizable liquid crystal compound different from the compound.

[23] The polymerizable composition as described in [21] or [22], further comprising a polymerization initiator.

[24] A cured product obtained by curing the polymerizable composition as described in any one of [21] to [23].

[25] An optical film comprising the cured product as described in [24].

[26] A polarizing plate comprising:

the optical film as described in [25]; and

a polarizer.

[27] An image display device comprising the optical film as described in [25] or the polarizing plate as described in [26].

According to the present invention, it is possible to provide a compound having a wide temperature range exhibiting liquid crystallinity and excellent precipitation suppression and solubility; and a polymerizable composition, a cured product, an optical film, a polarizing plate, and an image display device, each using the compound.

### BRIEF DESCRIPTION OF THE DRAWINGS

FIG. 1A is a schematic cross-sectional view showing an example of the optical film of the present invention.

FIG. 1B is a schematic cross-sectional view showing an example of the optical film of the present invention.

FIG. 1C is a schematic cross-sectional view showing an example of the optical film of the present invention.

### DESCRIPTION OF THE PREFERRED EMBODIMENTS

Hereinafter, the present invention will be described in detail.

Descriptions on the configuration requirements which will be described later are made based on representative embodiments of the present invention in some cases, but it should not be construed that the present invention is limited to such embodiments.

Furthermore, in the present specification, a numerical value range expressed using “to” means a range that includes the preceding and succeeding numerical values of “to” as the lower limit value and the upper limit value, respectively.

In addition, in the present specification, only one kind of the substance corresponding to each component may be used alone or two or more kinds thereof may also be used in combination, for each component. Here, in a case where the two or more substances are used in combination for each component, the content of the component refers to a total content of the substances used in combination unless otherwise specified.

[Compound]

The compound of an embodiment of the present invention is a compound represented by Formula (1) (hereinafter also simply referred to as a “compound (1)”) or a compound represented by Formula (2) (hereinafter also simply referred to as a “compound (2)”) which is other than the compound represented by Formula (1).

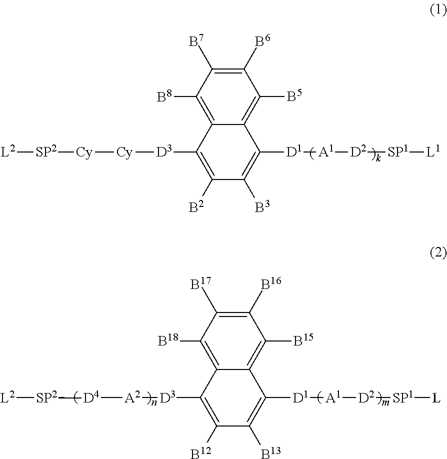

In the present invention, the compound having a structure represented by Formula (1) or (2) is a compound having a wide temperature range exhibiting liquid crystallinity and excellent precipitation suppression and solubility, as mentioned above.

A reason thereof is not specifically clear, but is presumed to be as follows by the present inventors.

That is, it is considered that by incorporating a naphthalene skeleton having a side chain structure at the first and fourth positions in the center (core) of the molecule, an interaction between the cores was enhanced and stacking properties between the molecules were improved, whereby the upper limit temperature exhibiting liquid crystallinity was increased and the temperature range was widened. In addition, it is considered that by specifying the type of a substituent in a case where the naphthalene skeleton had the substituent, the solubility was improved and the precipitation was suppressed.

Hereinafter, the compound (I) and the compound (II) will be described in detail.

[Compound (1)]

The compound (1) is a compound represented by Formula (1).

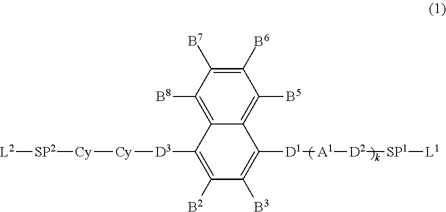

In Formula (1), A¹ represents an aromatic ring which may have a substituent or an alicyclic ring which may have a substituent.

Furthermore, in Formula (1), Cy represents a 1,4-cyclohexylene group which may have a substituent, and the two Cy's may be the same as or different from each other.

Moreover, in Formula (1), D¹, D², and D³ each independently represent a single bond, or a divalent linking group consisting of —O—, —CO—, —S—, —C(═S)—, —CR¹R²—, —CR¹═CR²—, —NR¹—, or a combination of two or more thereof, and R¹ and R² each independently represent a hydrogen atom, a fluorine atom, or an alkyl group having 1 to 4 carbon atoms.

Furthermore, in Formula (1), SP¹ and SP² each independently represent a single bond, a linear or branched alkylene group having 1 to 12 carbon atoms, or a divalent linking group obtained by each independently substituting one or more of —CH₂-'s constituting a linear or branched alkylene group having 1 to 12 carbon atoms with —O—, —CO—, —S—, —C(═S)—, —CR¹R²—, —CR¹═CR²—, or —NR¹—, and R¹ and R² each independently represent a hydrogen atom, a fluorine atom, or an alkyl group having 1 to 4 carbon atoms.

In addition, in Formula (1), k represents an integer of 1 to 3. In a case where k is 2 or 3, a plurality of A¹'s and D²'s which are present in the formula may be the same as or different from each other.

Furthermore, in Formula (1), L¹ and L² each independently represent a monovalent organic group, and at least one of L¹ or L² represents a polymerizable group.

In addition, in Formula (1), B², B³, B⁵, B⁶, B⁷, and B⁸ each independently represent a hydrogen atom or a substituent. It should be noted that in a case where at least one of B², B³, B⁶, or B⁷ represents a substituent, the substituent does not include a ring structure.

In Formula (1), examples of the aromatic ring shown in one aspect of A¹ include aromatic hydrocarbon rings such as a benzene ring, a naphthalene ring, an anthracene ring, and a phenanthroline ring; and aromatic heterocycles such as a furan ring, a thiophene ring, a pyrrole ring, an oxazole ring, an isoxazole ring, an oxadiazole ring, a thiazole ring, an isothiazole ring, a thiadiazole ring, an imidazole ring, a pyrazole ring, a triazole ring, a furazan ring, a tetrazole ring, a pyridine ring, a pyridazine ring, a pyrimidine ring, a pyrazine ring, a triazole ring, a tetrazine ring, and a benzothiazole ring. Among these, the benzene ring (for example, a 1,4-phenyl group) is preferable.

Moreover, examples of the alicyclic ring shown in one aspect of A¹ include cycloalkane rings such as a cyclohexane ring, a cyclopeptane ring, a cyclooctane ring, a cyclododecane ring, and a cyclodocosane ring. Among those, the cyclohexane ring (for example, a 1,4-cyclohexylene group) is preferable.

In addition, with respect to A¹, examples of the substituent which may be contained in the aromatic ring or the alicyclic ring include an alkyl group, an alkoxy group, and a halogen atom.

As the alkyl group, for example, a linear, branched, or cyclic alkyl group having 1 to 18 carbon atoms is preferable, an alkyl group having 1 to 8 carbon atoms (for example, a methyl group, an ethyl group, a propyl group, an isopropyl group, an n-butyl group, an isobutyl group, a sec-butyl group, a t-butyl group, and a cyclohexyl group) is more preferable, an alkyl group having 1 to 4 carbon atoms is still more preferable, and the methyl group or the ethyl group is particularly preferable.

As the alkoxy group, for example, an alkoxy group having 1 to 18 carbon atoms is preferable, an alkoxy group having 1 to 8 carbon atoms (for example, a methoxy group, an ethoxy group, an n-butoxy group, and a methoxyethoxy group) is more preferable, an alkoxy group having 1 to 4 carbon atoms is still more preferable, and the methoxy group or the ethoxy group is particularly preferable.

Examples of the halogen atom include a fluorine atom, a chlorine atom, a bromine atom, and an iodine atom, and among these, the fluorine atom or the chlorine atom is preferable.

In the present invention, A¹ in Formula (1) is preferably the alicyclic ring, more preferably the cycloalkane ring, still more preferably the cyclohexane ring, and particularly preferably the 1,4-cyclohexylene group.

In Formula (1), D¹, D², and D³ each independently represent a single bond, or a divalent linking group consisting of —O—, —CO—, —S—, —C(═S)—, —CR¹R²—, —CR¹═CR²—, —NR¹—, or a combination of two or more thereof, and R¹ and R² each independently represent a hydrogen atom, a fluorine atom, or an alkyl group having 1 to 4 carbon atoms.

For a reason of easy synthesis, among those, the substituent is preferably the single bond, —CO—, —COO—, —OCO—, —CO—NH—, or —NH—CO—, more preferably —COO—, and still more preferably —OCO—.

In addition, in a case where k is 2, it is preferable that D² present between two A¹'s is the single bond.

In addition, in a case where k is 3, it is preferable that at least one of D² present between a plurality of A¹'s is the single bond.

Suitable examples of the linear or branched alkylene group having 1 to 12 carbon atoms shown in one aspect of SP¹ and SP² in Formula (1) include a methylene group, an ethylene group, a propylene group, a butylene group, a pentylene group, a hexylene group, a methylhexylene group, and a heptylene group.

In addition, SP¹ and SP² may be a single bond, a linear or branched alkylene group having 1 to 12 carbon atoms, or a divalent linking group obtained by each independently substituting one or more of —CH₂-'s constituting a linear or branched alkylene group having 1 to 12 carbon atoms with —O—, —CO—, —S—, —C(═S)—, —CR¹R²—, —CR¹═CR²—, or —NR¹—, as described above, and R¹ and R² each independently represent a hydrogen atom, a fluorine atom, or an alkyl group having 1 to 4 carbon atoms. It should be noted that all of —CH₂-'s to be substituted are not —CH₂— constituting the alkylene group. In addition, in a case where —CH₂— is substituted with —O—, two consecutive —CH₂-'s are not substituted with —O—.

In Formula (1), k represents an integer of 1 to 3, and is preferably 2 or 3, and more preferably 2. Further, in a case where k is 2 or 3, a plurality of A¹'s and D²'s which are present in the formula may be the same as or different from each other.

In Formula (1), examples of the monovalent organic group represented by each of L¹ and L² include an alkyl group, an aryl group, and a heteroaryl group. The alkyl group may be linear, branched, or cyclic, but is preferably linear. The number of carbon atoms of the alkyl group is preferably 1 to 30, more preferably 1 to 20, and still more preferably 1 to 10. Further, the aryl group may be a monocycle or a polycycle, but is preferably the monocycle. The number of carbon atoms of the aryl group is preferably 6 to 25, and more preferably 6 to 10. Further, the heteroaryl group may be a monocycle or a polycycle. The number of heteroatoms constituting the heteroaryl group is preferably 1 to 3. The heteroatoms constituting the heteroaryl group is preferably a nitrogen atom, a sulfur atom, or an oxygen atom. The number of carbon atoms of the heteroaryl group is preferably 6 to 18, and more preferably 6 to 12. In addition, the alkyl group, the aryl group, and the heteroaryl group may be unsubstituted or have a substituent. Examples of the substituent include the same ones as the substituents which may be contained in A¹ in Formula (1).

On the other hand, in Formula (1), the polymerizable group represented by at least one of L¹ or L² is not particularly limited, but is preferably a polymerizable group which is radically polymerizable or cationically polymerizable.

A generally known radically polymerizable group can be used as the radically polymerizable group, and suitable examples thereof include an acryloyl group and a methacryloyl group. In this case, it is known that the acryloyl group generally has a high polymerization rate, and from the viewpoint of improvement of productivity, the acryloyl group is preferable but the methacryloyl group can also be used in the same manner as the polymerizable group.

A generally known cationically polymerizable group can be used as the cationically polymerizable group, and specific examples thereof include an alicyclic ether group, a cyclic acetal group, a cyclic lactone group, a cyclic thioether group, a spiroorthoester group, and a vinyloxy group. Among those, the alicyclic ether group or the vinyloxy group is suitable, and an epoxy group, an oxetanyl group, or the vinyloxy group is particularly preferable.

Particularly preferred examples of the polymerizable group include the following groups.

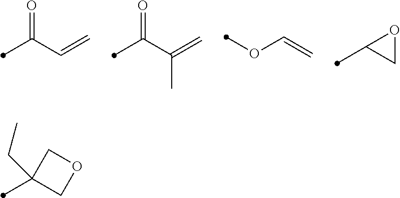

Among those, for a reason that the durability is improved, both of L¹ and L² in Formula (1) are preferably a polymerizable group, and more preferably an acryloyl group or a methacryloyl group.

In Formula (1), B², B³, B⁵, B⁶, B⁷, and B⁸ each independently represent a hydrogen atom or a substituent. It should be noted that in a case where at least one of B², B³, B⁶, or B⁷ represents a substituent, the substituent does not include a ring structure.

In the present invention, for a reason that the precipitation suppression and the solubility are improved, it is preferable that at least one of B², B³, B⁵, B⁶, B⁷, or B⁸ in Formula (1) represents a substituent (hereinafter also simply referred to as a “substituent B”).

Here, examples of the substituent B include an alkyl group, an alkoxy group, an alkylcarbonyl group, an alkoxycarbonyl group, an alkylcarbonyloxy group, an alkylamino group, a dialkylamino group, an alkylamide group, an alkenyl group, an alkynyl group, a halogen atom, a cyano group, a nitro group, an alkylthiol group, an N-alkylcarbamate group, an aryl group, an aryloxy group, an arylcarbonyl group, an arylcarbonyloxy group, an arylamino group, an arylamide group, an arylthiol group, an N-arylcarbamate group, a cycloalkyl group, a cycloalkyloxy group, a cycloalkylcarbonyl group, a cycloalkylcarbonyloxy group, a cycloalkylamino group, a cycloalkylamide group, a cycloalkylthiol group, an N-cycloalkylcarbamate group, a sulfonic acid ester group, or a monovalent organic group obtained by each independently substituting one or more of —CH₂-'s constituting an alkyl group with —O— or —CO—.

Furthermore, in a case where at least one of B², B³, B⁶, or B⁷ is a substituent, the substituent does not include a ring structure, and examples of the substituent B include an alkyl group, an alkoxy group, an alkylcarbonyl group, an alkoxycarbonyl group, an alkylcarbonyloxy group, an alkylamino group, a dialkylamino group, an alkylamide group, an alkenyl group, an alkynyl group, a halogen atom, a cyano group, a nitro group, an alkylthiol group, an N-alkylcarbamate group, and a monovalent organic group obtained by substituting one or more —CH₂-'s constituting an alkyl group with —O— or —CO—.

In the present invention, among the examples of the substituent B, the alkyl group, the alkoxy group, the alkoxycarbonyl group, or the alkylcarbonyloxy group is preferable.

As the alkyl group, for example, a linear, branched, or cyclic alkyl group having 1 to 18 carbon atoms is preferable, an alkyl group having 1 to 8 carbon atoms (for example, a methyl group, an ethyl group, a propyl group, an isopropyl group, an n-butyl group, an isobutyl group, a sec-butyl group, a t-butyl group, and a cyclohexyl group) is more preferable, an alkyl group having 1 to 4 carbon atoms is still more preferable, and the methyl group or the ethyl group is particularly preferable.

As the alkoxy group, for example, an alkoxy group having 1 to 18 carbon atoms is preferable, an alkoxy group having 1 to 8 carbon atoms (for example, a methoxy group, an ethoxy group, an n-butoxy group, and a methoxyethoxy group) is more preferable, an alkoxy group having to 4 carbon atoms is still more preferable, and the methoxy group or the ethoxy group is particularly preferable.

Examples of the alkoxycarbonyl group include a group in which an oxycarbonyl group (—O—CO— group) is bonded to the alkyl group exemplified above, and for example, the alkoxycarbonyl group is preferably a methoxycarbonyl group, an ethoxycarbonyl group, an n-propoxycarbonyl group, or an isopropoxycarbonyl group, and more preferably the methoxycarbonyl group.

Examples of the alkylcarbonyloxy group include a group in which a carbonyloxy group (—CO—O— group) is bonded to the alkyl group exemplified above, and for example, the alkylcarbonyloxy group is preferably a methylcarbonyloxy group, an ethylcarbonyloxy group, an n-propylcarbonyloxy group, or an isopropylcarbonyloxy group, and more preferably the methylcarbonyloxy group.

In the present invention, for a reason that the precipitation suppression and the solubility are particularly good, it is preferable that at least one of B⁵ or B⁸ in Formula (1) represents a substituent, and it is more preferable that B², B³, B⁶, and B⁷ in Formula (1) each represent a hydrogen atom.

In the present invention, for a reason that the precipitation suppression and the solubility are improved, it is preferable that at least one of B², B³, B⁶, or B⁷ in Formula (1) represents a substituent.

In the present invention, for a reason that the precipitation suppression and the solubility are particularly good, it is preferable that at least one of B² or B³ in Formula (1) represents a substituent, and it is more preferable that B⁵, B⁶, B⁷, and B⁸ in Formula (1) each represent a hydrogen atom.

In the present invention, for a reason that the precipitation suppression and the solubility are particularly good, it is preferable that at least one of B⁶ or B⁷ in Formula (1) represents a substituent, and it is more preferable that B², B³, B⁵, and B⁸ in Formula (1) each represent a hydrogen atom.

Specific examples of the compound (1) include compounds (1-1) to compounds (1-17) represented by the following formulae. Moreover, since a group adjacent to the acryloyloxy group in the structure of the compound (1-14) represents a propylene group (a group obtained by substituting a methyl group with an ethylene group), the compound 1-14 represents a mixture of regioisomers in which the positions of the methyl groups are different.

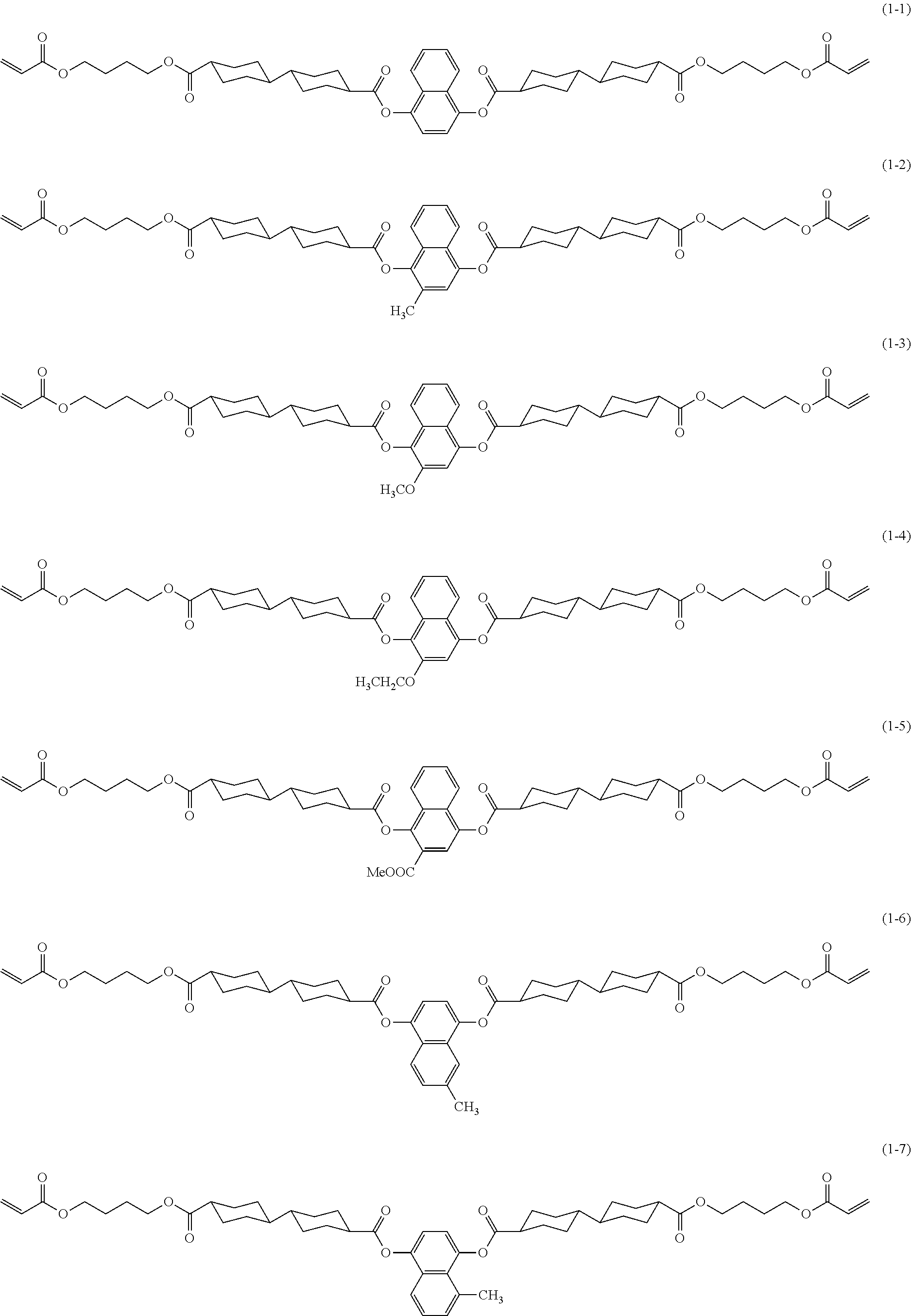

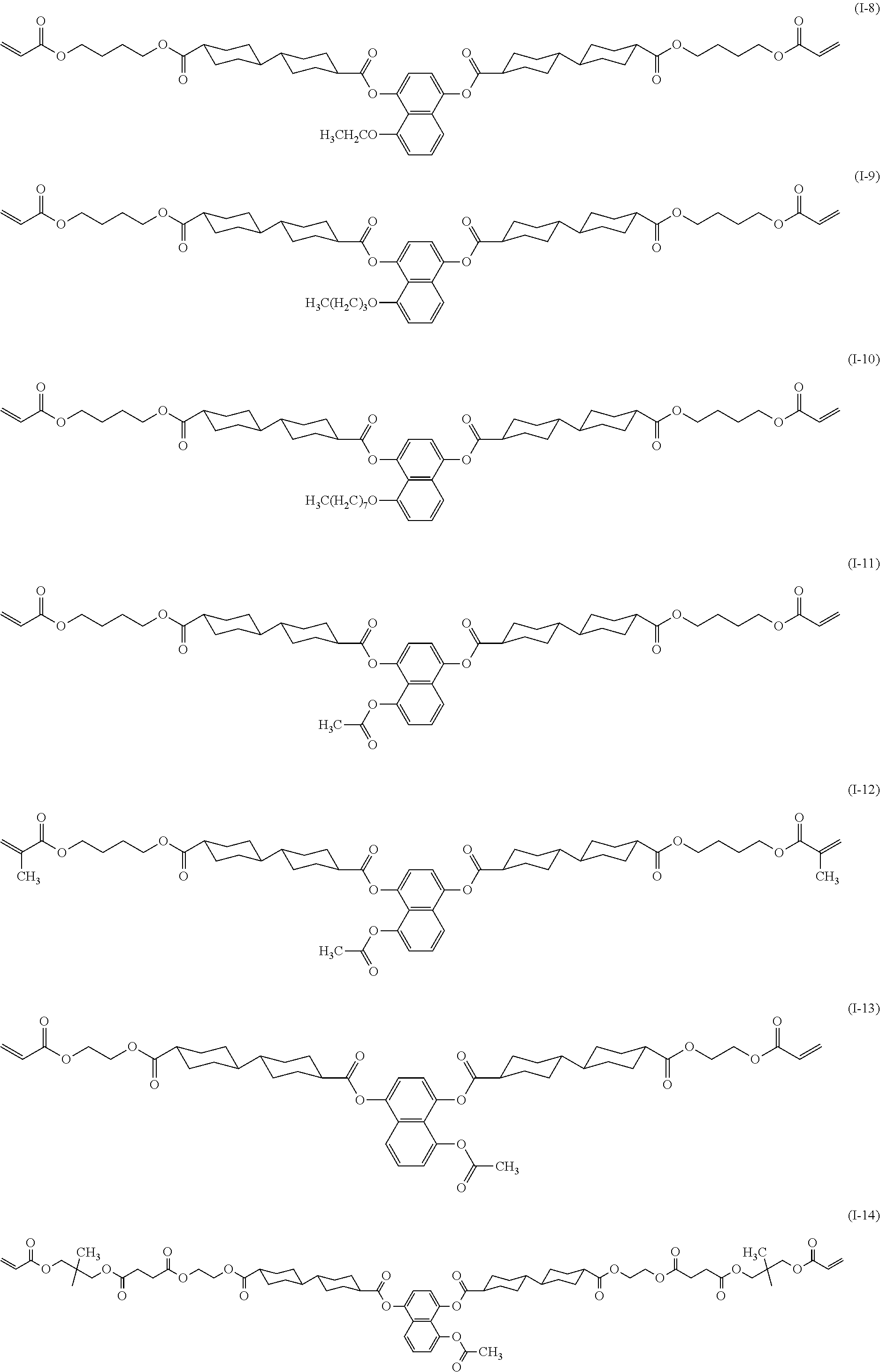

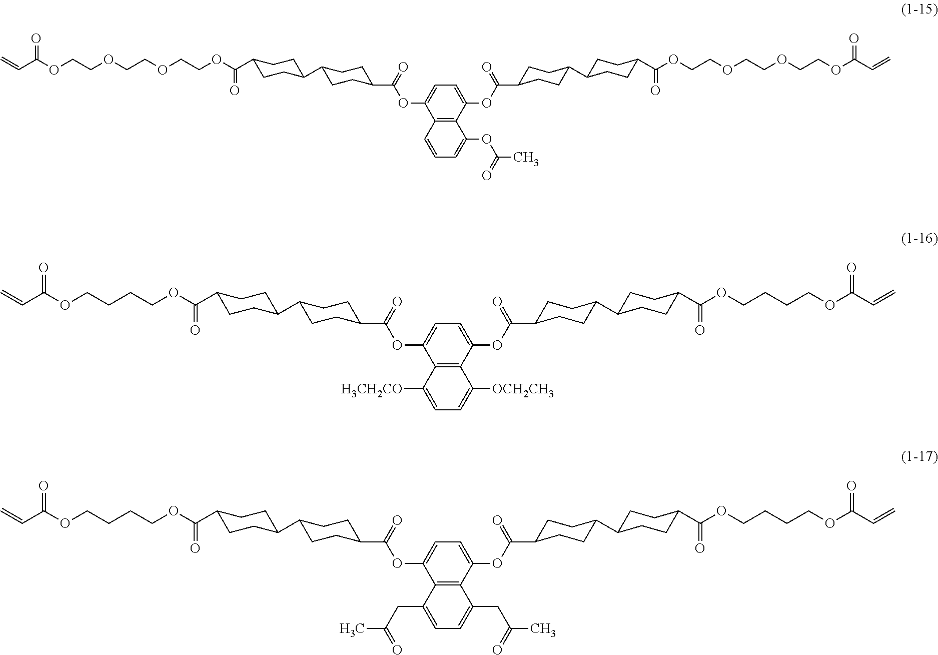

[Compound (2)]

The compound (2) is a compound obtained by removing the compound represented by Formula (1) from the compound represented by Formula (2). In other words, in the present specification, the compound corresponding to Formula (1) is referred to as a compound (1), and the compound not corresponding to Formula (1) but corresponding to Formula (2) is referred to as a compound (2).

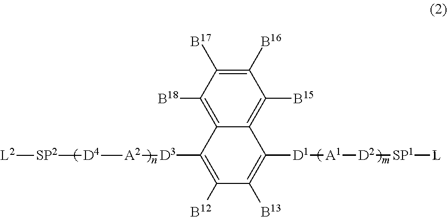

In Formula (2), A¹ and A² each independently represent an aromatic ring which may have a substituent or an alicyclic ring which may have a substituent.

Moreover, in Formula (2), D¹, D², D³, and D⁴ each independently represent a single bond, or a divalent linking group consisting of —O—, —CO—, —S—, —C(═S)—, —CR¹R²—, —CR¹—CR²—, —NR¹—, or a combination of two or more thereof, and R¹ and R² each independently represent a hydrogen atom, a fluorine atom, or an alkyl group having 1 to 4 carbon atoms.

Moreover, in Formula (2), SP¹ and SP² each independently represent a single bond, a linear or branched alkylene group having 1 to 12 carbon atoms, or a divalent linking group obtained by each independently substituting one or more of —CH₂-'s constituting a linear or branched alkylene group having 1 to 12 carbon atoms with —O—, —CO—, —S—, —C(═S)—, —CR¹R²—, —CR¹═CR²—, or —NR¹—, and R¹ and R² each independently represent a hydrogen atom, a fluorine atom, or an alkyl group having 1 to 4 carbon atoms.

Furthermore, in Formula (2), m and n each independently represent an integer of 1 to 3, and satisfy m+n=an integer of 3 to 6. In a case where m is 2 or 3, A¹'s and D²'s which are present in the formula may be the same as or different from each other. In a case where n is 2 or 3, A²'s and D⁴'s which are present in the formula may be the same as or different from each other.

Furthermore, in Formula (2), L¹ and L² each independently represent a monovalent organic group, and at least one of L¹ or L² represents a polymerizable group.

In addition, in Formula (2), B¹², B¹³, B¹⁵, B¹⁶, B¹⁷, and B¹⁸ each independently represent a hydrogen atom or a substituent. It should be noted that in a case where at least one of B¹², B¹³, B¹⁶, or B¹⁷ represents a substituent, the substituent does not include a ring structure, and in a case where at least one of B¹² or B¹³ represents a substituent, the substituent does not include —CHO.

In Formula (2), A¹ and A² are the same as A¹ described in Formula (1). Further, suitable aspects of A¹ and A² in Formula (2) are preferably the same as the suitable aspects of A¹ in Formula (1), that is, an aromatic ring that is not limited to an alicyclic ring.

In Formula (2), D¹, D², D³, and D⁴ each represent a single bond, or a divalent linking group consisting of —O—, —CO—, —S—, —C(═S)—, —CR¹R²—, —CR¹═CR²—, —NR¹—, or a combination of two or more thereof, and R¹ and R² each independently represent a hydrogen atom, a fluorine atom, or an alkyl group having 1 to 4 carbon atoms.

For a reason of easy synthesis, among those, the substituent is preferably the single bond, —CO—, —COO—, —OCO—, —CO—NH—, or —NH—CO—, more preferably —COO—, and still more preferably —OCO—.

Furthermore, in a case where m is 2 or 3, it is preferable that D² present between a plurality of A¹'s is —COO— or —OCO—.

In addition, in a case where n is 2 or 3, it is preferable that D⁴ present between a plurality of A²'s is —COO— or —OCO—.

In Formula (2), SP¹ and SP² are the same as SP¹ and SP² described in Formula (1), respectively.

In Formula (1), k, m, and n each independently represent an integer of 1 to 3, and satisfy m+n=an integer of 3 to 6.

m and n are each independently preferably 2 or 3, and more preferably 2. Further, in a case where m is 2 or 3, A¹'s and D²'s which are present in the formula may be the same as or different from each other. Similarly, in a case where n is 2 or 3, a plurality of A²'s and D⁴'s which are present in the formula may be the same as or different from each other.

In Formula (2), L¹ and L² are the same as L¹ and L² described in Formula (1), respectively.

In Formula (2), B¹², B¹³, B¹⁵, B¹⁶, B¹⁷, and B¹⁸ each independently represent a hydrogen atom or a substituent. It should be noted that in a case where at least one of B¹², B¹³, B¹⁶, or B¹⁷ represents a substituent, the substituent does not include a ring structure, and in a case where at least one of B¹² or B¹³ represents a substituent, the substituent does not include —CHO.

In the present invention, for a reason that the precipitation suppression and the solubility are improved, it is preferable that at least one of B¹², B¹³, B¹⁵, B¹⁶, B¹⁷, or B¹⁸ in Formula (2) represents a substituent.

Here, examples of the substituent include the same ones as the substituent B described for B¹ and the like in Formula (1).

Furthermore, in a case where at least one of B², B¹³, B¹⁶, or B¹⁷ is a substituent, the substituent does not include a ring structure, and examples of the substituent B include an alkyl group, an alkoxy group, an alkylcarbonyl group, an alkoxycarbonyl group, an alkylcarbonyloxy group, an alkylamino group, a dialkylamino group, an alkylamide group, an alkenyl group, an alkynyl group, a halogen atom, a cyano group, a nitro group, an alkylthiol group, an N-alkylcarbamate group, a sulfonic acid ester group, and a monovalent organic group obtained by substituting one or more —CH₂-'s constituting an alkyl group with —O— or —CO—.

In the present invention, for a reason that the precipitation suppression and the solubility are particularly good, it is preferable that at least one of B¹⁵ or B¹⁸ in Formula (2) represents a substituent, and it is more preferable that B¹², B¹³, B¹⁶, and B¹⁷ in Formula (2) each represent a hydrogen atom.

In the present invention, for a reason that the precipitation suppression and the solubility are improved, it is preferable that at least one of B¹², B¹³, B¹⁶, or B¹⁷ in Formula (2) represents a substituent.

In the present invention, for a reason that the precipitation suppression and the solubility are particularly good, it is preferable that at least one of B¹² or B¹³ in Formula (2) represents a substituent, and it is more preferable that B¹⁵, B¹⁶, B¹⁷, and B¹⁸ in Formula (2) each represent a hydrogen atom.

In the present invention, for a reason that the precipitation suppression and the solubility are particularly good, it is preferable that at least one of B¹⁶ or B¹⁷ in Formula (2) represents a substituent, and it is more preferable that B¹², B¹³, B¹⁵, and B¹⁸ in Formula (2) each represent a hydrogen atom.

Specific examples of the compound (2) include a compound (2-1) and a compound (2-2) represented by the following formulae.

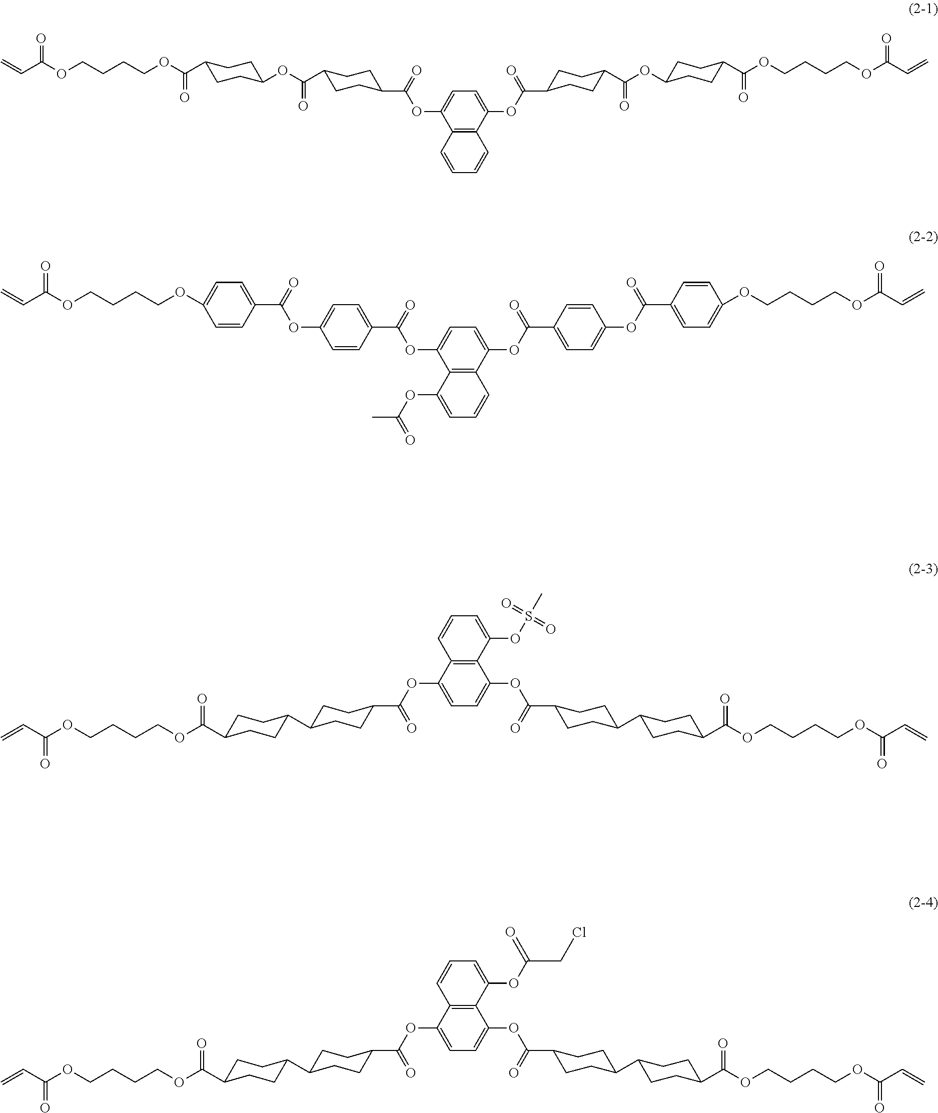

[Polymerizable Composition]

The polymerizable composition of an embodiment of the present invention is a polymerizable composition containing the above-mentioned compound of the embodiment of the present invention.

[Polymerizable Liquid Crystal Compound]

The polymerizable composition of the embodiment of the present invention preferably contains a polymerizable liquid crystal compound different from the above-mentioned compound of the embodiment of the present invention.

Here, the polymerizable liquid crystal compound means a liquid crystal compound having a polymerizable group.

The liquid crystal compounds can be generally classified into a rod-shaped type and a disk-shaped type according to the shape thereof. Each of the types can further be classified into a low-molecular-weight type and a high-molecular-weight type. The expression, being high-molecular, generally refers to having a degree of polymerization of 100 or more (Polymer Physics-Phase Transition Dynamics, by Masao Doi, page 2, published by Iwanami Shoten, Publishers, 1992).

In the present invention, any of liquid crystal compounds can be used, but a rod-shaped liquid crystalline compound or a discotic liquid crystalline compound (disk-shaped liquid crystalline compound) is more preferably used.

The polymerizable liquid crystal compound preferably has two or more polymerizable groups in one molecule from the viewpoint of immobilization of the above-mentioned liquid crystal compound.

The type of the polymerizable group is not particularly limited, a functional group capable of performing an addition polymerization reaction is preferable, and an ethylenically unsaturated polymerizable group or a polymerizable ring group is more preferable. More specifically, preferred examples of the polymerizable group include an acryloyl group, a methacryloyl group, a vinyl group, a styryl group, and an allyl group, and the acryloyl group or the methacryloyl group is more preferable.

As the rod-shaped liquid crystalline compound, for example, the rod-shaped liquid crystalline compounds described in claim 1 of JP1999-513019A (JP-H11-513019A) or paragraphs [0026] to [0098] of JP2005-289980A can be preferably used, and as the discotic liquid crystal compound, for example, the discotic liquid crystalline compounds described in paragraphs [0020] to [0067] of JP2007-108732A and paragraphs [0013] to [0108] of JP2010-244038A can be preferably used, but the rod-shaped liquid crystalline compound is not limited thereto.

In the present invention, a reverse wavelength dispersible liquid crystal compound can be used as the polymerizable liquid crystal compound.

Here, in the present specification, the “reverse wavelength dispersible” liquid crystal compound means that in a case where an in-plane retardation (Re) value at a specific wavelength (visible light range) of a phase difference film manufactured using the polymerizable liquid crystal compound is measured, the Re value is equal or higher as a measurement wavelength is increased.

In addition, the reverse wavelength dispersible liquid crystal compound is not particularly limited as long as it can form the reverse wavelength dispersible film as described above, and for example, the compound represented by General Formula (I) described in JP2008-297210A (in particular, the compounds described in paragraph Nos. [0034] to [0039]), the compounds represented by General Formula (I) described in JP2010-084032A (in particular, the compounds described in paragraph Nos. [0067] to [0073]), the compound represented by General Formula (II) described in JP2016-053709A (in particular, the compounds described in paragraph Nos. [0036] to [0043]), the compounds represented by General Formula (1) described in JP2016-081035A (in particular, the compounds described in paragraph Nos. [0043] to [0055]), or the like can be used.

In addition, for a reason that the reverse wavelength dispersibility is improved, suitable examples of the polymerizable liquid crystal compound include compounds represented by Formulae (1) to (10), and specifically include the compounds having side chain structures shown in Table 1 and 2 below as K (side chain structure) in Formulae (1) to (10).

Furthermore, in Tables 1 and 2 below, “*” shown in the side chain structure of K represents a bonding position to an aromatic ring.

In addition, in the side chain structures shown in 1-2 in Table 1 below and 2-2 in Table 2 below, a group adjacent to each of the acryloyloxy group and the methacryloyl group represents a propylene group (a group in which a methyl group is substituted with an ethylene group), and represents a mixture of regioisomers in which the positions of the methyl groups are different.

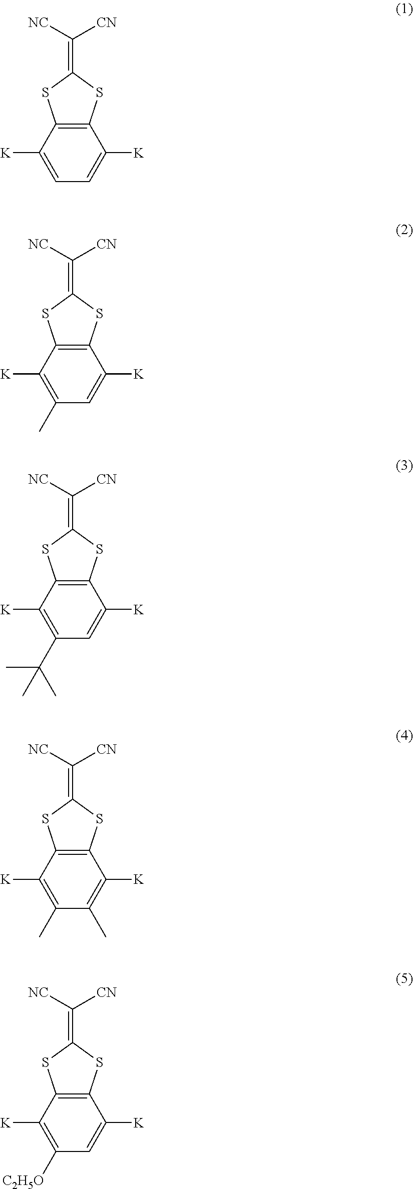

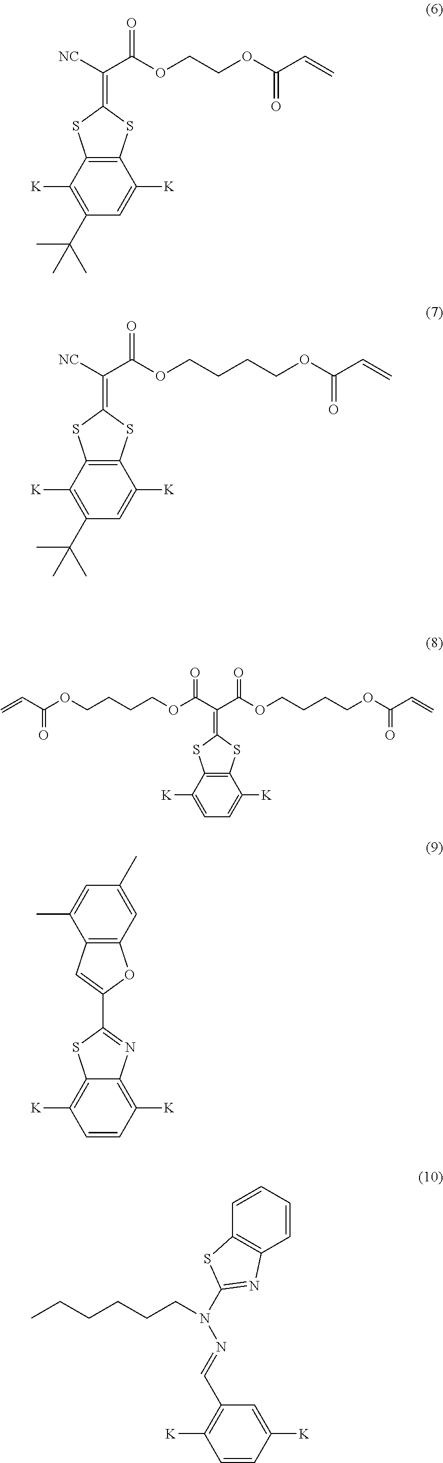

TABLE 1

| Table 1 | K (side chain structure) |
|---|---|
| 1-1 | 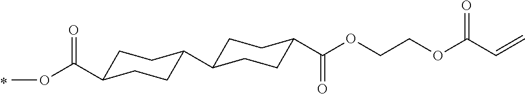 [SMILES: C=CC(=O)OCCOC(=O)C1CCC(C2CCC(C(=O)OC)CC2)CC1] |
| 1-2 | 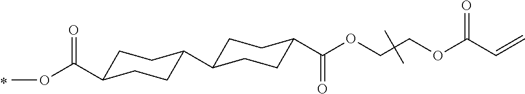 [SMILES: C=CC(=O)OCC(C)(C)COC(=O)C1CCC(C2CCC(C(=O)OC)CC2)CC1] |
| 1-3 | 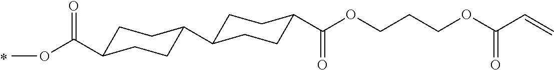 [SMILES: C=CC(=O)OCCCOC(=O)C1CCC(C2CCC(C(=O)OC)CC2)CC1] |
| 1-4 | 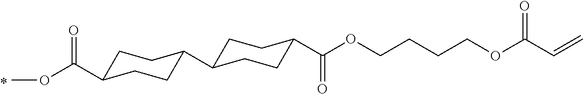 [SMILES: C=CC(=O)OCCCCOC(=O)C1CCC(C2CCC(C(=O)OC)CC2)CC1] |
| 1-5 | 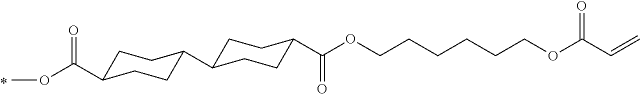 [SMILES: C=CC(=O)OCCCCCCOC(=O)C1CCC(C2CCC(C(=O)OC)CC2)CC1] |
| 1-6 | 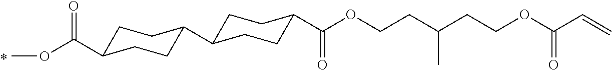 [SMILES: C=CC(=O)OCCC(C)CCOC(=O)C1CCC(C2CCC(C(=O)OC)CC2)CC1] |
| 1-7 | 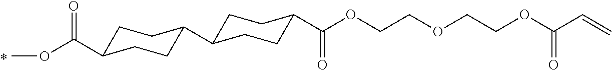 [SMILES: C=CC(=O)OCCOCCOC(=O)C1CCC(C2CCC(C(=O)OC)CC2)CC1] |
| 1-8 | 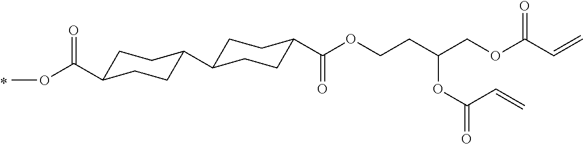 [SMILES: C=CC(=O)OCC(CCOC(=O)C1CCC(C2CCC(C(=O)OC)CC2)CC1)OC(=O)C=C] |
| 1-9 | 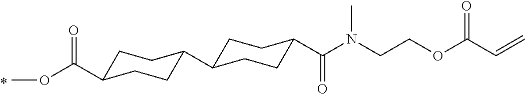 [SMILES: C=CC(=O)OCCN(C)C(=O)C1CCC(C2CCC(C(=O)OC)CC2)CC1] |
| 1-10 | 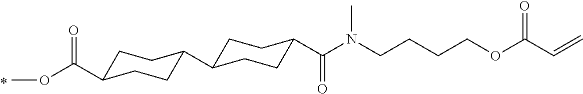 [SMILES: C=CC(=O)OCCCCN(C)C(=O)C1CCC(C2CCC(C(=O)OC)CC2)CC1] |
| 1-11 | 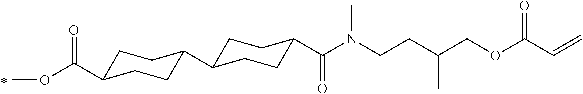 [SMILES: C=CC(=O)OCC(C)CCN(C)C(=O)C1CCC(C2CCC(C(=O)OC)CC2)CC1] |
| 1-12 | 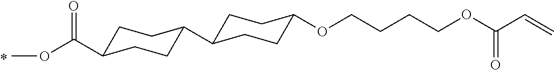 [SMILES: C=CC(=O)OCCCCOC1CCC(C2CCC(C(=O)OC)CC2)CC1] |
| 1-13 | 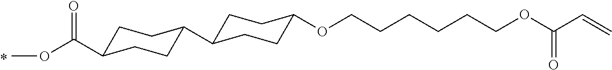 [SMILES: C=CC(=O)OCCCCCCOC1CCC(C2CCC(C(=O)OC)CC2)CC1] |

TABLE 2

| Table 2 | K (side chain structure) |
|---|---|
| 2-1 | 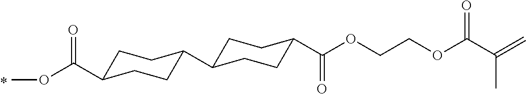 [SMILES: C=C(C)C(=O)OCCOC(=O)C1CCC(C2CCC(C(=O)OC)CC2)CC1] |
| 2-2 | 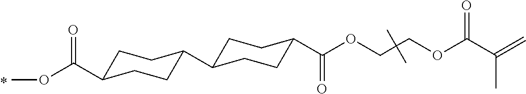 [SMILES: C=C(C)C(=O)OCC(C)(C)COC(=O)C1CCC(C2CCC(C(=O)OC)CC2)CC1] |
| 2-3 | 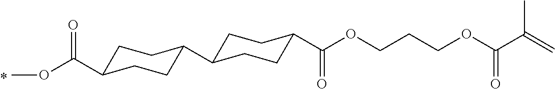 [SMILES: C=C(C)C(=O)OCCCOC(=O)C1CCC(C2CCC(C(=O)OC)CC2)CC1] |
| 2-4 | 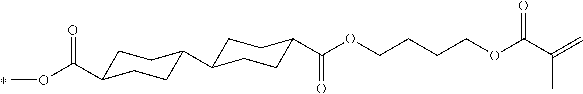 [SMILES: C=C(C)C(=O)OCCCCOC(=O)C1CCC(C2CCC(C(=O)OC)CC2)CC1] |
| 2-5 | 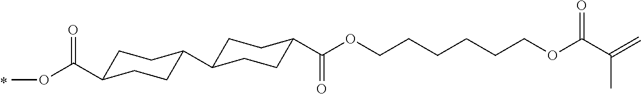 [SMILES: C=C(C)C(=O)OCCCCCCOC(=O)C1CCC(C2CCC(C(=O)OC)CC2)CC1] |
| 2-6 | 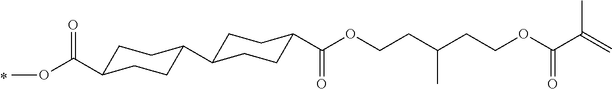 [SMILES: C=C(C)C(=O)OCCC(C)CCOC(=O)C1CCC(C2CCC(C(=O)OC)CC2)CC1] |
| 2-7 | 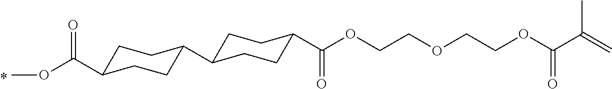 [SMILES: C=C(C)C(=O)OCCOCCOC(=O)C1CCC(C2CCC(C(=O)OC)CC2)CC1] |
| 2-8 | 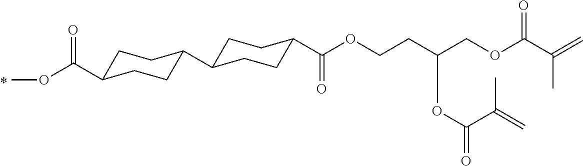 [SMILES: C=C(C)C(=O)OCC(CCOC(=O)C1CCC(C2CCC(C(=O)OC)CC2)CC1)OC(=O)C(=C)C] |
| 2-9 | 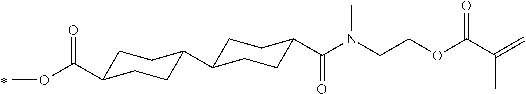 [SMILES: C=C(C)C(=O)OCCN(C)C(=O)C1CCC(C2CCC(C(=O)OC)CC2)CC1] |
| 2-10 | 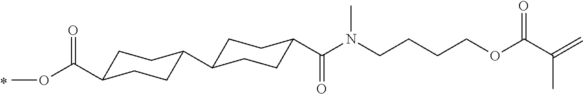 [SMILES: C=C(C)C(=O)OCCCCN(C)C(=O)C1CCC(C2CCC(C(=O)OC)CC2)CC1] |
| 2-11 | 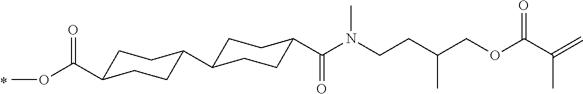 [SMILES: C=C(C)C(=O)OCC(C)CCN(C)C(=O)C1CCC(C2CCC(C(=O)OC)CC2)CC1] |
| 2-12 | 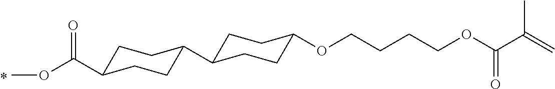 [SMILES: C=C(C)C(=O)OCCCCOC1CCC(C2CCC(C(=O)OC)CC2)CC1] |
| 2-13 | 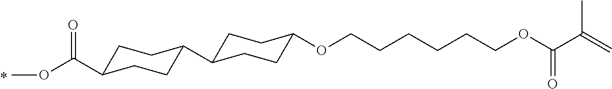 [SMILES: C=C(C)C(=O)OCCCCCCOC1CCC(C2CCC(C(=O)OC)CC2)CC1] |

[Polymerization Initiator]

The polymerizable composition of the embodiment of the present invention preferably contains a polymerization initiator.

The polymerization initiator to be used is preferably a photopolymerization initiator capable of initiating a polymerization reaction upon irradiation with ultraviolet rays.

Examples of the photopolymerization initiator include α-carbonyl compounds (described in each of the specifications of U.S. Pat. Nos. 2,367,661A and 2,367,670A), acyloin ethers (described in the specification of U.S. Pat. No. 2,448,828A), α-hydrocarbon-substituted aromatic acyloin compounds (described in the specification of U.S. Pat. No. 2,722,512A), multinuclear quinone compounds (described in each of the specifications of U.S. Pat. Nos. 3,046,127A and 2,951,758A), combinations of a triarylimidazole dimer and a p-aminophenyl ketone (described in the specification of U.S. Pat. No. 3,549,367A), acridine and phenazine compounds (described in JP1985-105667A (JP-S60-105667A) and the specification of U.S. Pat. No. 4,239,850A), oxadiazole compounds (described in the specification of U.S. Pat. No. 4,212,970A), and acyl phosphine oxide compounds (described in JP1988-40799B (JP-S63-40799B), JP1993-29234B (JP-H05-29234B), JP1998-95788A (JP-H10-95788A), and JP1998-29997A (JP-H10-29997A)).

In addition, in the present invention, it is also preferable that the polymerization initiator is an oxime-type polymerization initiator, and specific examples of the polymerization initiator include the initiators described in paragraphs [0049] to [0052] of WO2017/170443A.

[Solvent]

It is preferable that the polymerizable composition of the embodiment of the present invention contains a solvent from the viewpoint of workability for forming a cured product (for example, an optically anisotropic layer) of an embodiment of the present invention, which will be described later.

Specific examples of the solvent include ketones (for example, acetone, 2-butanone, methyl isobutyl ketone, cyclohexanone, and cyclopentanone), ethers (for example, dioxane and tetrahydrofuran), aliphatic hydrocarbons (for example, hexane), alicyclic hydrocarbons (for example, cyclohexane), aromatic hydrocarbons (for example, toluene, xylene, and trimethylbenzene), halogenated carbons (for example, dichloromethane, dichloroethane, dichlorobenzene, and chlorotoluene), esters (for example, methyl acetate, ethyl acetate, and butyl acetate), water, alcohols (for example, ethanol, isopropanol, butanol, and cyclohexanol), cellosolves (for example, methyl cellosolve and ethyl cellosolve), cellosolve acetates, sulfoxides (for example, dimethyl sulfoxide), and amides (for example, dimethylformamide and dimethylacetamide), and these may be used singly or in combination of two or more kinds thereof.

[Leveling Agent]

It is preferable that the polymerizable composition of the embodiment of the present invention contains a leveling agent from the viewpoint that the surface of a cured product of an embodiment of the present invention, which will be described later, is maintained smooth and the alignment is easily controlled.

Such a leveling agent is preferably a fluorine-based leveling agent or a silicon-based leveling agent for a reason that it has a high leveling effect on the addition amount, and the leveling agent is more preferably a fluorine-based leveling agent from the viewpoint that it is less likely to cause bleeding (bloom or bleed).

Specific example of the leveling agent include the compounds described in paragraphs [0079] to [0102] of JP2007-069471A, the compound represented by General Formula (I) described in JP2013-047204A (in particular, the compounds described in paragraphs [0020] to [0032]), the compound represented by General Formula (1) described in JP2012-211306A (in particular, the compounds described in paragraphs [0022] to [0029]), the liquid crystal alignment accelerator represented by General Formula (1) described in JP2002-129162A (in particular, the compounds described in paragraphs [0076] to [0078] and [0082] to [0084]), and the compounds represented by General Formulae (I), (II), and (III) described in JP2005-099248A (in particular, the compounds described in paragraphs [0092] to [0096]). In addition, the leveling agent may also function as an alignment control agent which will be described later.

[Alignment Control Agent]

The polymerizable composition of the embodiment of the present invention can contain an alignment control agent, as desired.

With the alignment control agent, various alignment states such as homeotropic alignment (vertical alignment), tilt alignment, hybrid alignment, and cholesteric alignment can be formed, in addition to the homogeneous alignment, and specific alignment states can be controlled and achieved more uniformly and more accurately.

As an alignment control agent which accelerates the homogeneous alignment, for example, a low-molecular-weight alignment control agent or a high-molecular-weight alignment control agent can be used.

With regard to the low-molecular-weight alignment control agent, reference can be made to the description in, for example, paragraphs [0009] to [0083] of JP2002-20363A, paragraphs [0111] to [0120] of JP2006-106662A, and paragraphs [0021] to [0029] of JP2012-211306A, the contents of which are hereby incorporated by reference.

In addition, with regard to the high-molecular-weight alignment control agent, reference can be made to the description in, for example, paragraphs [0021] to [0057] of JP2004-198511A and paragraphs [0121] to [0167] of JP2006-106662A, the contents of which are hereby incorporated by reference.

Furthermore, examples of the alignment control agent that forms or accelerates the homeotropic alignment include a boronic acid compound and an onium salt compound, and specifically, reference can be made to the compounds described in paragraphs [0023] to [0032] of JP2008-225281A, paragraphs [0052] to [0058] of JP2012-208397A, paragraphs [0024] to [0055] of JP2008-026730A, paragraphs [0043] to [0055] of JP2016-193869A, and the like, the contents of which are hereby incorporated by reference.

On the other hand, the cholesteric alignment can be achieved by adding a chiral agent to the composition of the embodiment of the present invention, and it is possible to control the direction of revolution of the cholesteric alignment by its chiral direction.

Incidentally, it is possible to control the pitch of the cholesteric alignment in accordance with the alignment regulating force of the chiral agent.

In a case where an alignment control agent is contained, a content thereof is preferably 0.01% to 10% by mass, and more preferably 0.05% to 5% by mass with respect to the mass of the total solid content of the composition. In a case where the content is within the range, it is possible to obtain a cured product which has no precipitation or phase separation, alignment defects, or the like, and is uniform and highly transparent while achieving a desired alignment state.

These alignment control agents can further impart a polymerizable functional group, in particular, a polymerizable functional group that is polymerizable with the compound (I) included in the composition of the embodiment of the present invention.

[Other Components]

The polymerizable composition of the embodiment of the present invention may contain components other than the above-mentioned components, and examples of such other components include a surfactant, a tilt angle control agent, an alignment assistant, a plasticizer, and a crosslinking agent.

[Cured Product]

The cured product of the embodiment of the present invention is a cured product obtained by curing the above-mentioned polymerizable composition of the embodiment of the present invention.

Here, in a case where the polymerizable composition of the embodiment of the present invention contains, for example, a polymerizable liquid crystal compound different from the above-mentioned compound (I) together with the compound (I), it is possible to form an optically anisotropic layer as a cured product by polymerizing the polymerizable composition of the embodiment of the present invention.

Examples of a method for forming the cured product include a method in which the above-mentioned polymerizable composition of the embodiment of the present invention is used to cause a desired alignment state, and then fixed by polymerization.

Here, the polymerization conditions are not particularly limited, but in the polymerization by irradiation with light, ultraviolet rays are preferably used. The irradiation dose is preferably 10 mi/cm² to 50 J/cm², more preferably 20 mJ/cm² to 5 J/cm², still more preferably 30 mJ/cm² to 3 J/cm², and particularly preferably 50 mJ/cm² to 1,000 mJ/cm². In addition, the polymerization may be carried out under a heating condition in order to accelerate the polymerization reaction.

In addition, in the present invention, the cured product can be formed on any of supports in the optical film of the embodiment of the present invention, which will be described later or a polarizer in the polarizing plate of an embodiment of the present invention, which will be described later.

The cured product of the embodiment of the present invention is preferably an optically anisotropic layer satisfying Formula (I).

0.50<Re(450)/Re(550)<1.00 (I)

Here, in Formula (I), Re(450) represents an in-plane retardation at a wavelength of 450 nm of the optically anisotropic layer, and Re(550) represents an in-plane retardation at a wavelength of 550 nm of the optically anisotropic layer. In addition, in the present specification, in a case where the measurement wavelength of the retardation is not specified, the measurement wavelength is 550 nm.

Furthermore, the values of the in-plane retardation and the thickness-direction retardation refer to values measured with light at the measurement wavelength using AxoScan OPMF-1 (manufactured by Opto Science, Inc.).

Specifically, by inputting the average refractive index ((Nx+Ny+Nz)/3) and the film thickness (d (μm)) to AxoScan OPMF-1, it is possible to calculate:

Slow axis direction (°)

Re(λ)=R0(λ)

Rth(λ)=((nx+ny)/2−nz)×d.

In addition, R0(λ) is expressed in a numerical value calculated with AxoScan OPMF-1, but means Re(λ).

In addition, such an optically anisotropic layer is preferably a positive A-plate or a positive C-plate, and more preferably the positive A-plate.

Here, the positive A-plate (A-plate which is positive) and the positive C-plate (C-plate which is positive) are defined as follows.

In a case where a refractive index in a film in-plane slow axis direction (in a direction in which an in-plane refractive index is a maximum) is defined as nx, a refractive index in an in-plane direction orthogonal to the in-plane slow axis is defined as ny, and a refractive index in a thickness direction is defined as nz, the positive A-plate satisfies the relationship of Formula (A1) and the positive C-plate satisfies the relationship of Formula (C1). In addition, the positive A-plate has an Rth showing a positive value and the positive C-plate has an Rth showing a negative value.

nx>ny≈nz Formula (A1)

nz>nx≈ny Formula (C1)

Furthermore, the symbol, “≈”, encompasses not only a case where the both are completely the same as each other but also a case where the both are substantially the same as each other.

The expression, “substantially the same”, means that with regard to the positive A-plate, for example, a case where (ny−nz)×d (in which d is the thickness of a film) is −10 to 10 nm, and preferably −5 to 5 nm is also included in “ny≈nz”, and a case where (nx−nz)×d is −10 to 10 nm, and preferably −5 to 5 nm is also included in “nx≈nz”. In addition, with regard to the positive C-plate, for example, a case where (nx−ny)×d (in which d is the thickness of a film) is 0 to 10 nm, and preferably 0 to 5 nm is also included in “nx≈ny”.

In a case where the optically anisotropic layer is a positive A-plate, the Re(550) is preferably 100 to 180 nm, more preferably 120 to 160 nm, still more preferably 130 to 150 nm, and particularly preferably 130 to 140 nm, from the viewpoint that the optically anisotropic layer functions as a λ/4 plate.

Here, the “λ/4 plate” is a plate having a λ/4 function, specifically, a plate having a function of converting a linearly polarized light at a certain specific wavelength into a circularly polarized light (or converting a circularly polarized light to a linearly polarized light).

[Optical Film]

The optical film of the embodiment of the present invention is an optical film having the cured product of the embodiment of the present invention.

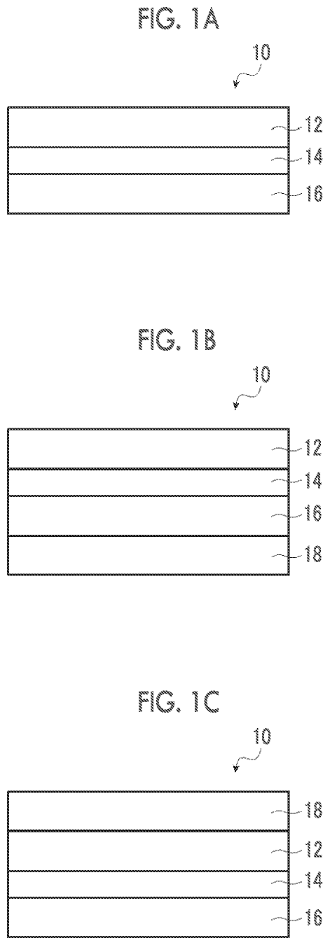

FIG. 1A, FIG. 1B, and FIG. 1C (these drawings are hereinafter simply referred to as “FIG. 1” unless it is necessary that they are particularly distinguished from each other) are each a schematic cross-sectional view showing an example of the optical film of the embodiment of the present invention.

Furthermore, FIG. 1 is a schematic view, and the thicknesses relationship, the positional relationship, and the like among the respective layers are not necessarily consistent with actual ones, and any of the support, the alignment film, and the hard coat layer shown in FIG. 1 are optional constitutional members.

An optical film 10 shown in FIG. 1 has a support 16, an alignment film 14, and an optically anisotropic layer 12 as the cured product in this order.

In addition, the optical film 10 may have a hard coat layer 18 on the side of the support 16 opposite to the side on which the alignment film 14 is provided as shown in FIG. 1B, and may have the hard coat layer 18 on the side of the optically anisotropic layer 12 opposite to the side on which the alignment film 14 is provided as shown in FIG. 1C.

Hereinafter, various members used for the optical film of the embodiment of the present invention will be described in detail.

[Cured Product]

The cured product contained in the optical film of the embodiment of the present invention is the above-mentioned cured product of the embodiment of the present invention.

In the optical film of the embodiment of the present invention, the thickness of the cured product is not particularly limited, but in a case where the optical film functions as an optically anisotropic layer, the thickness of the cured product is preferably 0.1 to 10 μm, and more preferably 0.5 to 5 μm.

[Support]

The optical film of the embodiment of the present invention may have a support as a base material for forming a cured product as described above.

Such a support is preferably transparent, and specifically, it preferably has a light transmittance of 80% or more.

Examples of such a support include a glass substrate and a polymer film, and examples of the material for the polymer film include cellulose-based polymers; acrylic polymers having an acrylic ester polymer such as polymethyl methacrylate and a lactone ring-containing polymer, thermoplastic norbornene-based polymers; polycarbonate-based polymers; polyester-based polymers such as polyethylene terephthalate and polyethylene naphthalate; styrene-based polymers such as polystyrene and an acrylonitrile-styrene copolymer (AS resin); polyolefin-based polymers such as polyethylene, polypropylene, and an ethylene-propylene copolymer; vinyl chloride-based polymers; amide-based polymers such as nylon and aromatic polyamide; imide-based polymers; sulfone-based polymers; polyether sulfone-based polymers; polyether ether ketone-based polymers; polyphenylene sulfide-based polymers; vinylidene chloride-based polymers; vinyl alcohol-based polymers; vinyl butyral-based polymers; arylate-based polymers; polyoxymethylene-based polymers; epoxy-based polymers; and polymers obtained by mixing these polymers.

In addition, an aspect in which a polarizer which will be described later may also function as such a support is also available.

In the present invention, the thickness of the support is not particularly limited, but is preferably 5 to 60 μm, and more preferably 5 to 30 μm.

[Alignment Film]

In a case where the optical film of the embodiment of the present invention has any of the above-mentioned supports, it is preferable that the optical film has an alignment film between the support and the cured product. Further, an aspect in which the above-mentioned support may also function as an alignment film is also available.

The alignment film generally has a polymer as a main component. Polymer materials for an alignment film are described in many documents, and many commercially available products can be used.

The polymer material used in the present invention is preferably a polyvinyl alcohol or a polyimide, or a derivative thereof. Particularly, a modified or non-modified polyvinyl alcohol is preferable.

Examples of the alignment film that can be used in the present invention include the alignment films described for Line 24 on Page 43 to Line 8 on Page 49 of WO01/88574A; the modified polyvinyl alcohols described in paragraphs [0071] to [0095] of JP3907735B; and the liquid crystal alignment film formed by a liquid crystal alignment agent described in JP2012-155308A.

In the present invention, for a reason that it is possible to prevent deterioration in the surface condition by avoiding a contact with a surface of an alignment film upon formation of the alignment film, a photo-alignment film is also preferably used as the alignment film.

The photo-alignment film is not particularly limited, but the polymer materials such as a polyamide compound and a polyimide compound, described in paragraphs 0024 to 0043 of WO2005/096041A; the liquid crystal alignment film formed by a liquid crystal alignment agent having a photo-alignment group, described in JP2012-155308A; LPP-JP265CP, trade name, manufactured by Rolic Technologies Ltd.; or the like can be used.

In addition, in the present invention, the thickness of the alignment film is not particularly limited, but from the viewpoint of forming an optically anisotropic layer having a uniform film thickness by alleviating the surface roughness that can be present on the support, the thickness is preferably 0.01 to 10 μm, more preferably 0.01 to 1 μm, and still more preferably 0.01 to 0.5 μm.

[Hard Coat Layer]

It is preferable that the optical film of the embodiment of the present invention has a hard coat layer in order to impart physical strength to the film. Specifically, the optical film may have the hard coat layer on the side of the support opposite to the side on which the alignment film is provided (see FIG. 1B) or the optical film may have the hard coat layer on the side of the optically anisotropic layer opposite to the side on which the alignment film is provided (see FIG. 1C).

As the hard coat layer, those described in paragraphs [0190] to [0196] of JP2009-98658A can be used.

[Ultraviolet Absorber]

The optical film of the embodiment of the present invention preferably includes an ultraviolet (UV) absorber, taking an effect of external light (particularly ultraviolet rays) into consideration.

The ultraviolet absorber may be contained in the cured product of the embodiment of the present invention or may also be contained in a member other than the cured product constituting the optical film of the embodiment of the present invention. Suitable examples of the member other than the cured product include a support.

As the ultraviolet absorber, any one of ultraviolet absorbers known in the related art, which can express ultraviolet absorptivity, can be used. Among such the ultraviolet absorbers, a benzotriazole-based or hydroxyphenyltriazine-based ultraviolet absorber is preferably used from the viewpoint that it has high ultraviolet absorptivity and ultraviolet absorbing ability (ultraviolet-shielding ability) used for an image display device is obtained.

In addition, in order to broaden ultraviolet absorbing ranges, two or more of ultraviolet absorbers having different maximum absorption wavelengths can be used in combination.

Specific examples of the ultraviolet absorber include the compounds described in paragraphs [0258] and [0259] of JP2012-18395A and the compounds described in paragraphs [0055] to [0105] of JP2007-72163A.

In addition, as a commercially available product thereof, for example, Tinuvin 400, Tinuvin 405, Tinuvin 460, Tinuvin 477, Tinuvin 479, and Tinuvin 1577 (all manufactured by BASF), or the like can be used.

[Polarizing Plate]

A polarizing plate of an embodiment of the present invention has the above-mentioned optical film of the embodiment of the present invention and a polarizer. Furthermore, in a case where the optically anisotropic layer as the above-mentioned cured product of the embodiment of the present invention is a λ/4 plate (positive A-plate), the polarizing plate of the embodiment of the present invention can be used as a circularly polarizing plate.

In addition, in a case where the optically anisotropic layer as the above-mentioned cured product of the embodiment of the present invention is a λ/4 plate (positive A-plate), an angle between the slow axis of the λ/4 plate and the absorption axis of a polarizer which will be described later is preferably 30° to 60°, more preferably 40° to 50°, still more preferably 42° to 48°, and particularly preferably 45° in the polarizing plate of the embodiment of the present invention.

Here, the “slow axis” of the λ/4 plate means a direction in which the refractive index in the plane of the λ/4 plate is a maximum, and the “absorption axis” of the polarizer means a direction in which the absorbance is highest.

[Polarizer]

A polarizer contained in a polarizing plate of an embodiment of the present invention is not particularly limited as long as it is a member having a function of converting light into specific linearly polarized light, and an absorptive type polarizer and a reflective type polarizer, which are known in the related art, can be used.

An iodine-based polarizer, a dye-based polarizer using a dichroic dye, a polyene-based polarizer, or the like is used as the absorptive type polarizer. The iodine-based polarizer and the dye-based polarizer are classified into a coating type polarizer and a stretching type polarizer, any of which can be applied, but a polarizer which is manufactured by allowing polyvinyl alcohol to adsorb iodine or a dichroic dye and performing stretching is preferable.

In addition, examples of a method of obtaining a polarizer by carrying out stretching and dyeing in a state of a laminated film in which a polyvinyl alcohol layer is formed on a base material include the methods disclosed in JP5048120B, JP5143918B, JP4691205B, JP4751481B, and JP4751486B, and known technologies relating to these polarizers can also be preferably used.

A polarizer in which thin films having different birefringence are laminated, a wire grid-type polarizer, a polarizer having a combination of a cholesteric liquid crystal having a selective reflection range and a ¼ wavelength plate, or the like is used as the reflective type polarizer.

Among those, a polymer containing a polyvinyl alcohol-based resin (—CH₂—CHOH— as a repeating unit) since of its superior adhesion. In particular, a polarizer containing at least one selected from the group consisting of polyvinyl alcohol and an ethylene-vinyl alcohol copolymer) is preferable.

In the present invention, the thickness of the polarizer is not particularly limited, but is preferably 3 μm to 60 μm, more preferably 5 μm to 30 μm, and still more preferably 5 μm to 15 μm.

[Pressure Sensitive Adhesive Layer]

The polarizing plate of the embodiment of the present invention may have a pressure sensitive adhesive layer arranged between the cured product in the optical film of the embodiment of the present invention and the polarizer.

The pressure sensitive adhesive layer used for lamination of the cured product and the polarizer represents, for example, a substance in which a ratio (tan δ=G″/G′) between a storage elastic modulus G′ and a loss elastic modulus G″, each measured with a dynamic viscoelastometer, is 0.001 to 1.5, and examples thereof include a so-called pressure sensitive adhesive or a readily creepable substance. Examples of the pressure sensitive adhesive that can be used in the present invention include a polyvinyl alcohol-based pressure sensitive adhesive, but the pressure sensitive adhesive is not limited thereto.

[Image Display Device]

An image display device of an embodiment of the present invention is an image display device having the optical film of the embodiment of the present invention or the polarizing plate of the embodiment of the present invention.

A display element used in the image display device of the embodiment of the present invention is not particularly limited, and examples thereof include a liquid crystal cell, an organic electroluminescent (hereinafter simply referred to as “EL”) display panel, and a plasma display panel.

Among those, the liquid crystal cell and the organic EL display panel are preferable, and the liquid crystal cell is more preferable. That is, as the image display device of the embodiment of the present invention, a liquid crystal display device using a liquid crystal cell as a display element or an organic EL display device using an organic EL display panel as a display element is preferable, and the liquid crystal display device is more preferable.

[Liquid Crystal Display Device]

A liquid crystal display device which is an example of the image display device of the embodiment of the present invention is a liquid crystal display device having the above-mentioned polarizing plate of the embodiment of the present invention and a liquid crystal cell.

In addition, in the present invention, it is preferable that the polarizing plate of the embodiment of the present invention is used as the polarizing plate of the front side, and it is more preferable that the polarizing plate of the embodiment of the present invention is used as the polarizing plates on the front and rear sides, among the polarizing plates provided on the both sides of the liquid crystal cell.

Hereinafter, the liquid crystal cell constituting the liquid crystal display device will be described in detail.

<Liquid Crystal Cell>

The liquid crystal cell used for the liquid crystal display device is preferably in a vertical alignment (VA) mode, an optically compensated bend (OCB) mode, an in-plane-switching (IPS) mode, or a twisted nematic (TN) mode, but is not limited thereto.

In a TN-mode liquid crystal cell, rod-shaped liquid crystal molecules are substantially horizontally aligned and are twist-aligned at 60° to 120° during no voltage application thereto. A TN-mode liquid crystal cell is most often used in a color TFT liquid crystal display device and described in numerous documents.

In a VA-mode liquid crystal cell, rod-shaped liquid crystal molecules are substantially vertically aligned during no voltage application thereto. Examples of the VA-mode liquid crystal cell include (1) a VA-mode liquid crystal cell in the narrow sense of the word, in which rod-shaped liquid crystal molecules are substantially vertically aligned during no voltage application thereto, but are substantially horizontally aligned during voltage application thereto (described in JP1990-176625A (JP-H02-176625A)), (2) an MVA-mode liquid crystal cell in which the VA mode is multi-domained for viewing angle enlargement (described in SID97, Digest of Tech. Papers (preprint), 28 (1997) 845), (3) a liquid crystal cell in a mode (n-ASM mode) in which rod-shaped liquid crystal molecules are substantially vertically aligned during no voltage application thereto and are multi-domain-aligned during voltage application thereto (described in Seminar of Liquid Crystals of Japan, Papers (preprint), 58-59 (1998)), and (4) a survival-mode liquid crystal cell (announced in LCD International 98). In addition, the liquid crystal cell may be of any of a patterned vertical alignment (PVA) type, an optical alignment type, and polymer-sustained alignment (PSA) type. Details of these modes are specifically described in JP2006-215326A and JP2008-538819A.

In an IPS-mode liquid crystal cell, rod-shaped liquid crystal molecules are aligned substantially parallel with respect to a substrate, and application of an electric field parallel to the substrate surface causes the liquid crystal molecules to respond planarly. The IPS mode displays black in a state where no electric field is applied and a pair of upper and lower polarizing plates have absorption axes which are orthogonal to each other. A method of improving the viewing angle by reducing light leakage during black display in an oblique direction using an optical compensation sheet is disclosed in JP1998-54982A (JP-H10-54982A), JP1999-202323A (JP-H11-202323A), JP1997-292522A (JP-H09-292522A), JP1999-133408A (JP-H11-133408A), JP1999-305217A (JP-H11-305217A), JP1998-307291A (JP-H10-307291A), and the like.

[Organic EL Display Device]

Suitable examples of the organic EL display device which is an example of the image display device of the embodiment of the present invention include an aspect which includes, from the visible side, a polarizer, a λ/4 plate (a positive A-plate) including the optically anisotropic layer of the embodiment of the present invention, and an organic EL display panel in this order.

Furthermore, the organic EL display panel is a display panel composed of an organic EL device in which an organic light emitting layer (organic electroluminescent layer) is sandwiched between electrodes (between a cathode and an anode). The configuration of the organic EL display panel is not particularly limited but a known configuration is adopted.

### EXAMPLES

Hereinafter, the present invention will be described in more detail with reference to Examples. The materials, the amounts of materials used, the proportions, the treatment details, the treatment procedure, and the like shown in Examples below can be appropriately modified as long as the modifications do not depart from the spirit of the present invention. Therefore, the scope of the present invention should not be construed as being limited to Examples shown below.

### Example 1

[Synthesis of Compound (1-1)]

<Synthesis of Carboxylic Acid Derivative (S-1-d)>

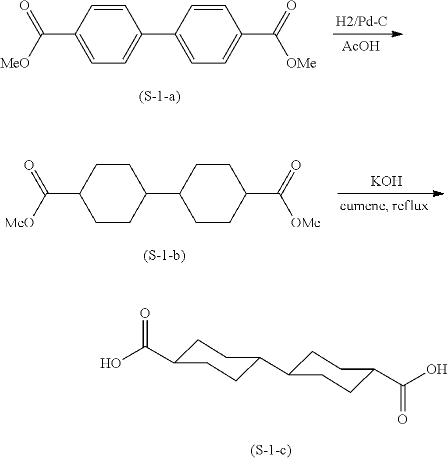 [SMILES: "AcOH" ; COC(=O)c1ccc(-c2ccc(C(=O)OC)cc2)cc1 ; COC(=O)C1CCC(C2CCC(C(=O)OC)CC2)CC1 ; "cumene, reflux" ; "KOH" ; O=C(O)C1CCC(C2CCC(C(=O)O)CC2)CC1]

As shown in the scheme, 125 g (0.462 mol) of dimethyl 4,4-biphenyl dicarboxylate (S-1-a) was added to 1,000 mL of acetic acid, 12.5 g of a palladium carbon catalyst (wet body) was added thereto, and the mixture was subjected to a catalytic hydrogenation reaction at 130° C. and 2 MPa in an autoclave.

After the completion of the reaction, the mixture was cooled to room temperature and the catalyst was removed by filtration. After evaporating acetic acid under reduced pressure, ethyl acetate and an aqueous sodium bicarbonate solution were added to the residue, the mixture was stirred and subjected to liquid separation to remove the aqueous layer, and the organic layer was washed with 10% physiological saline. The solution was dried by addition of sodium sulfate and the solvent was concentrated to obtain dimethyl 4,4′-dicyclohexane dicarboxylate (S-1-b) (130 g).

While not carrying out further purification, dimethyl 4,4′-dicyclohexane dicarboxylate (130 g), 86.3 g of potassium hydroxide pellets (manufactured by Aldrich, purity: 90%), 1,300 mL of cumene, and 10 mL of polyethylene glycol (PEG2000) were subsequently added thereto, and the mixture was mixed, and heated and stirred at 120° C. with a Dean-Stark tube. After evaporating methanol, the outside equipment was set to a temperature of 180° C., and heating and refluxing were continued for 20 hours while evaporating the solvent. The progress of the reaction was confirmed by nuclear magnetic resonance (NMR), and after the completion of the reaction, the reaction solution was cooled, 1,300 mL of ethanol was added thereto, and the precipitated potassium salt was collected by filtration.

Subsequently, this potassium salt was dissolved in 1,300 ml of water, concentrated hydrochloric acid was added thereto under ice-cooling until the pH of the system reached 3, and the precipitated carboxylic acid was collected by filtration to recover a crude product.

The recovered crude product was suspended in 500 mL of acetone, stirred at 50° C. for 30 minutes, and then cooled to room temperature to recover crystals by filtration. By repeating this reslurry operation twice, 93.9 g of crystals of dicyclohexanedicarboxylic acid (S-1-c) having a trans-form content of almost 100% were obtained (yield: 80%).

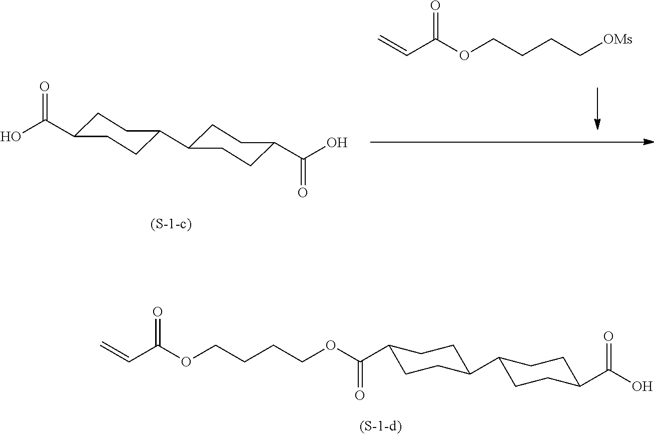 [SMILES: C=CC(=O)OCCCCOS(C)(=O)=O ; O=C(O)C1CCC(C2CCC(C(=O)O)CC2)CC1 ; C=CC(=O)OCCCCOC(=O)C1CCC(C2CCC(C(=O)O)CC2)CC1]

Subsequently, as shown in the scheme, 10.0 g (39.3 mmol) of the compound (S-1-c), 50 mL of N,N-dimethylacetamide (DMAc), 8.0 ml (78.6 mmol) of triethylamine, and 433 mg of 2,6-di-t-butyl-4-methylphenol were mixed at room temperature (23° C.).

To the mixture was added 9.61 g (43.2 mmol) of 4-methylsulfonyloxybutyl acrylate, and the mixture was stirred at 90° C. for 5 hours. The mixture was cooled to room temperature, then a mixed solution of 2.60 g of concentrated hydrochloric acid and 20 ml of water was added thereto, and the mixture was stirred at 40° C. and then subjected to liquid separation. Subsequently, to the organic layer were added 20 ml of toluene and 30 ml of a 5% aqueous sodium bicarbonate solution, and the mixture was stirred at 40° C. and subjected to liquid separation. Next, after washing the organic layer twice with 30 ml of a 1% aqueous sodium bicarbonate solution, 20 mg of 2,2,6,6-tetramethylpiperidin-1-oxyl (TEMPO) was added thereto and then the solvent was evaporated under reduced pressure. As converted by means of NMR and high performance liquid chromatography (HPLC), the content of the main product was 28%. This toluene solution was heated to 40° C., 45 mL of hexane was added thereto, the internal temperature was lowered to 5° C., and 12 mL of hexane was further added to the mixture. The mixture was stirred as it was for 10 minutes, and the solid was collected by filtration and washed with 30 mL of hexane. 8.5 mL of toluene and 55 mL of hexane were added to the obtained solid, the mixture was heated to 40° C. and then cooled to an internal temperature of 5° C., and reslurry washing was performed. The solid was collected by filtration and washed with 35 mL of hexane to obtain 6.7 g (yield 45%) of a compound (S-1-d).

<Synthesis of Compound (1-1)>

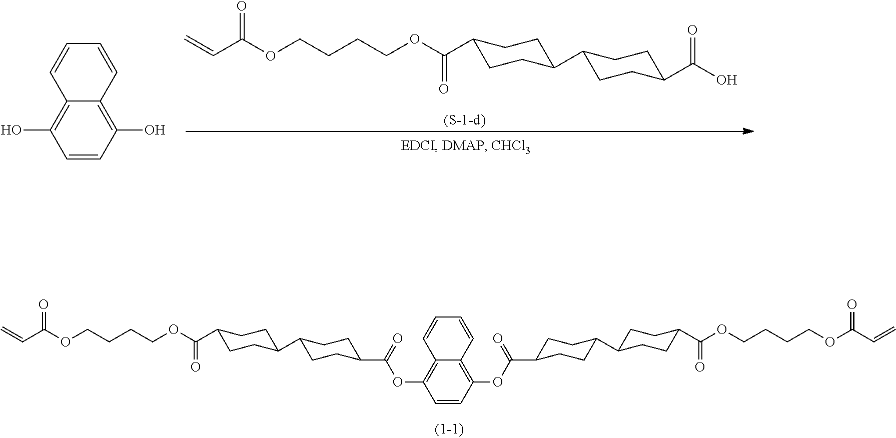 [SMILES: Oc1ccc(O)c2ccccc12 ; C=CC(=O)OCCCCOC(=O)C1CCC(C2CCC(C(=O)O)CC2)CC1 ; C=CC(=O)OCCCCOC(=O)C1CCC(C2CCC(C(=O)Oc3ccc(OC(=O)C4CCC(C5CCC(C(=O)OCCCCOC(=O)C=C)CC5)CC4)c4ccccc34)CC2)CC1]

0.5 g (3.12 mmol) of 1,4-dihydroxynaphthalene, 2.97 g (7.80 mmol) of the compound (S-1-d), 38 mg of 4-dimethylaminopyridine (DMAP), 35 mg of 2,6-di-t-butyl-4-methylphenol, and 5 mL of chloroform (CHCl₃) were mixed, 1.61 g (8.42 mmol) of 1-(3-dimethylaminopropyl)-3-ethylcarbodiimide (EDCI) hydrochloride was added thereto, and the mixture was stirred at room temperature for 2 hours. The reaction solution was purified as it was with a silica gel column to obtain 2.2 g (yield 79%) of a compound (1-1).

The ¹H-nuclear magnetic resonance (NMR) of the obtained compound (1-1) is shown below.

¹H-NMR (solvent: CDCl₃) δ (ppm): 7.89-7.80 (m, 2H), 7.58-7.48 (m, 2H), 7.20 (s, 2H), 6.42 (d, 2H), 6.12 (dd, 2H), 5.85 (d, 2H), 4.19 (t, 4H), 4.10 (t, 4H), 2.76-2.60 (m, 2H), 2.39-2.18 (m, 6H), 2.11-1.57 (m, 24H), 1.51-1.32 (m, 411), 1.30-0.99 (m, 12H)

### Example 2

[Synthesis of Compound (1-2)]

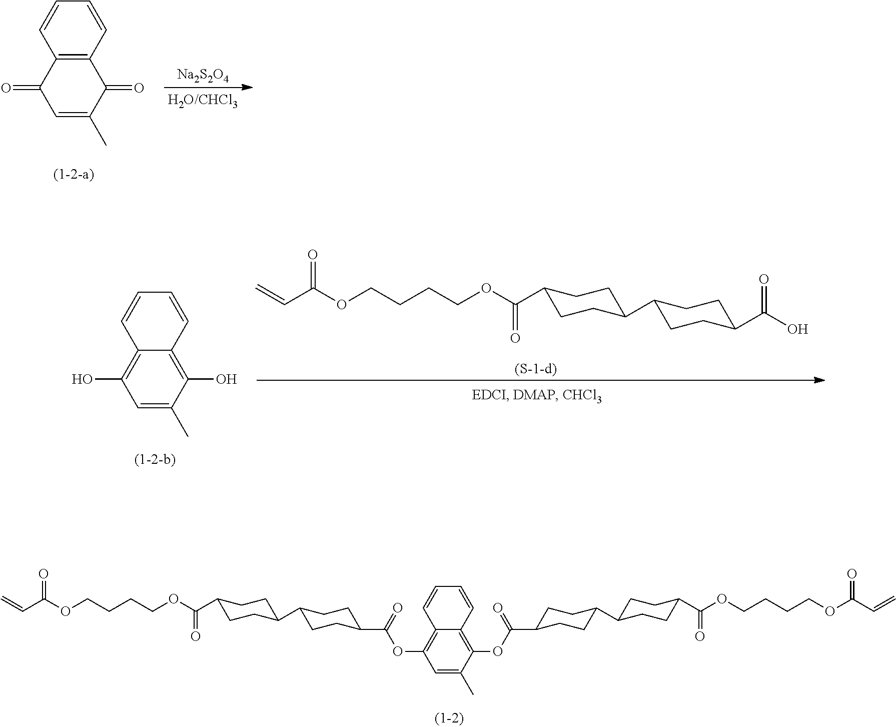

10.0 g (58.1 mmol) of 2-methyl-1,4-naphthoquinone (1-2-a) was dissolved in 160 mL of chloroform, and a solution obtained by mixing 20.2 g (116 mmol) of sodium hydrosulfite and 160 mL of water was added dropwise thereto under water-cooling. After the dropwise addition, the mixture was stirred at room temperature for 1 hour and the precipitated solid was collected by filtration. The solid was washed with chloroform and water to obtain 7.1 g (yield 70%) of 2-methyl-1,4-dihydroxynaphthalene. Next, 2.0 g (yield 72%) of a compound (1-2) was obtained by performing the reaction in the same manner as for the compound (1-1), using 0.50 g (3.12 mmol) of the compound (1-2-b).

The ¹H-NMR of the obtained compound (1-2) is shown below.

¹H-NMR (solvent: CDCl₃) δ (ppm): 7.78 (d, 1H), 7.70 (d, 1H), 6.12 (7.53-7.40 (m, 2H), 7.13 (s, 1H), 6.41 (d, 2H), 6.14 (dd, 2H), 5.82 (d, 2H), 4.20 (t, 4H), 4.10 (t, 4H), 2.73-2.58 (m, 2H), 2.37-2.18 (m, 9H), 2.06-0.99 (m, 40H)

### Example 3

[Synthesis of Compound (1-3)]

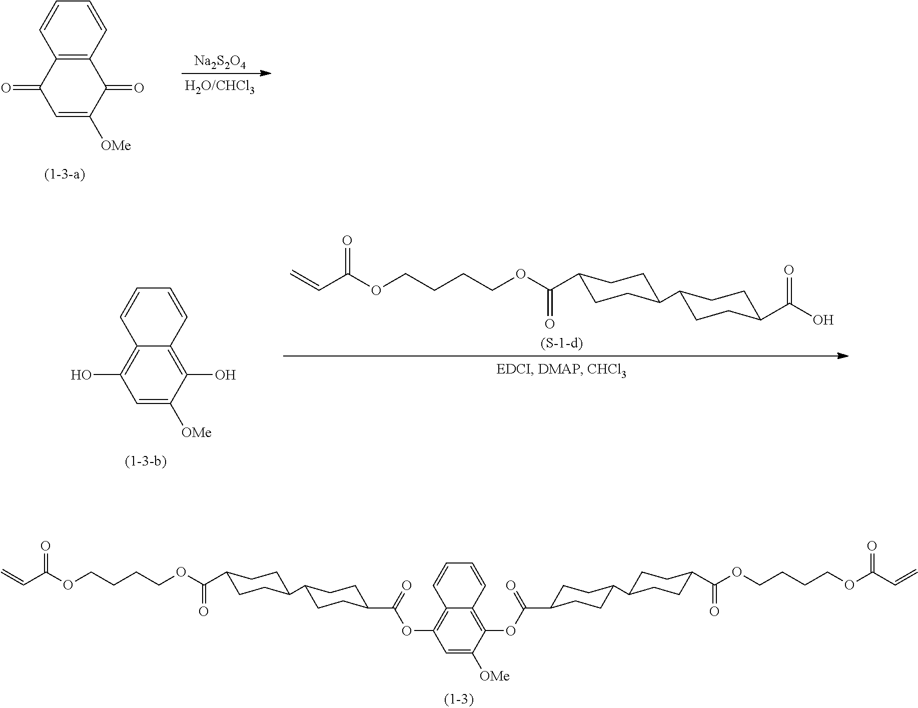 [SMILES: COC1=CC(=O)c2ccccc2C1=O ; "Na2S2O4" ; COc1cc(O)c2ccccc2c1O ; C=CC(=O)OCCCCOC(=O)C1CCC(C2CCC(C(=O)O)CC2)CC1 ; C=CC(=O)OCCCCOC(=O)C1CCC(C2CCC(C(=O)Oc3cc(OC)c(OC(=O)C4CCC(C5CCC(C(=O)OCCCCOC(=O)C=C)CC5)CC4)c4ccccc34)CC2)CC1]

2.9 g (yield 61%) of a compound (1-3) was obtained using 1.0 g (5.26 mmol) of the obtained compound (1-3-b) by the synthesis in the same manner as for the compound (1-2-b), except that the raw material was changed to 2-methoxy-1,4-naphthoquinone (1-3-a).

The ¹H-NMR of the obtained compound (1-3) is shown below.

¹H-NMR (solvent: CDCl₃) δ (ppm): 7.76 (t, 2H), 7.48 (t, 1H), 7.36 (t, 1H), 7.13 (s, 1H), 6.40 (d, 2H), 6.11 (dd, 2H), 5.84 (d, 2H), 4.20 (t, 4H), 4.10 (t, 4H), 3.91 (s, 3H), 2.75-2.59 (m, 2H), 2.37-2.16 (m, 6H), 2.09-0.97 (m, 40H)

### Example 4

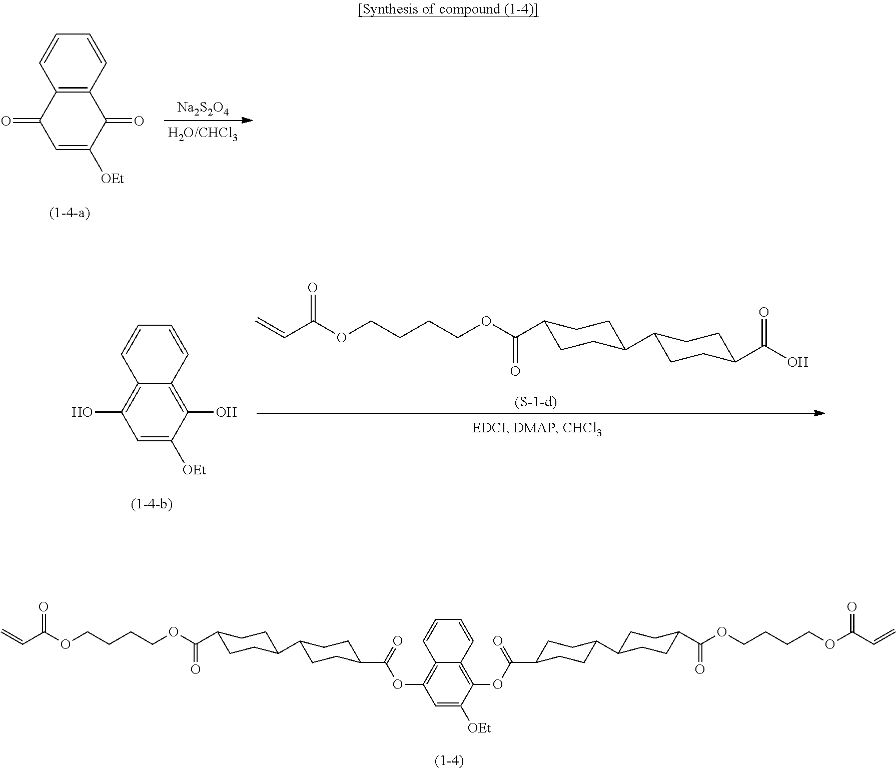

2.1 g (yield 58%) of a compound (1-4) was obtained using 0.8 g (3.92 mmol) of the obtained compound (1-4-b) by the synthesis in the same manner as for the compound (1-2-b), except that the raw material was changed to 2-ethoxy-1,4-naphthoquinone (1-4-a).

The ¹H-NMR of the obtained compound (1-4) is shown below.

¹H-NMR (solvent: CDCl₃) δ (ppm): 7.79-7.69 (m, 2H), 7.46 (t, 1H), 7.34 (t, 1H), 7.11 (s, 1H), 4.24-4.05 (m, 10H), 2.72-2.58 (m, 2H), 2.39-2.17 (m, 6H), 2.08-1.32 (m, 31H), 1.24-0.98 (m, 12H)

### Example 5

[Synthesis of Compound (1-5)]

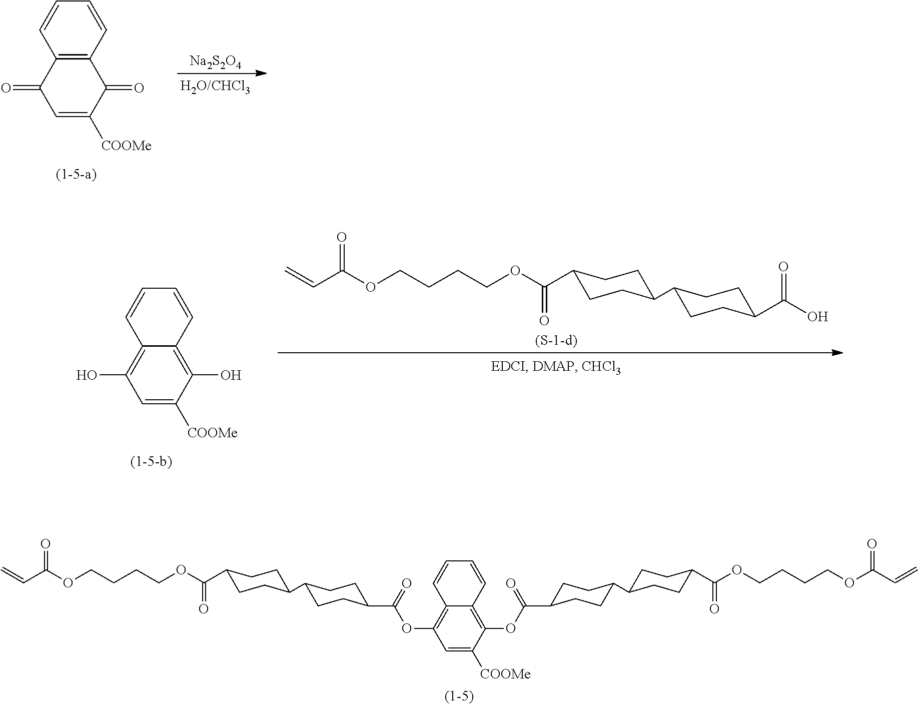 [SMILES: COC(=O)C1=CC(=O)c2ccccc2C1=O ; "Na2S2O4" ; COC(=O)c1cc(O)c2ccccc2c1O ; C=CC(=O)OCCCCOC(=O)C1CCC(C2CCC(C(=O)O)CC2)CC1 ; C=CC(=O)OCCCCOC(=O)C1CCC(C2CCC(C(=O)Oc3cc(C(=O)OC)c(OC(=O)C4CCC(C5CCC(C(=O)OCCCCOC(=O)C=C)CC5)CC4)c4ccccc34)CC2)CC1]

1.76 g (yield 51%) of a compound (1-5) was obtained using 0.8 g (3.67 mmol) of the obtained compound (1-5-b) by the synthesis in the same manner as for the compound (1-2-b), except that the raw material was changed to 2-methoxycarbonyl-1,4-naphthoquinone (1-5-a).

The ¹H-NMR of the obtained compound (1-5) is shown below.

¹H-NMR (solvent: CDCl₃) δ (ppm): 7.78 (d, 1H), 7.85 (d, 1H), 7.77 (s, 1H), 7.66-7.54 (m, 2H), 6.42 (d, 21H), 6.15 (dd, 2H), 5.84 (d, 2H), 4.21 (t, 4H), 4.10 (t, 4H), 3.90 (s, 3H), 2.79-2.61 (m, 2H), 2.44-2.19 (m, 6H), 2.05-1.34 (m, 28H), 1.25-0.99 (m, 12H)

### Example 6

[Synthesis of Compound (1-6)]

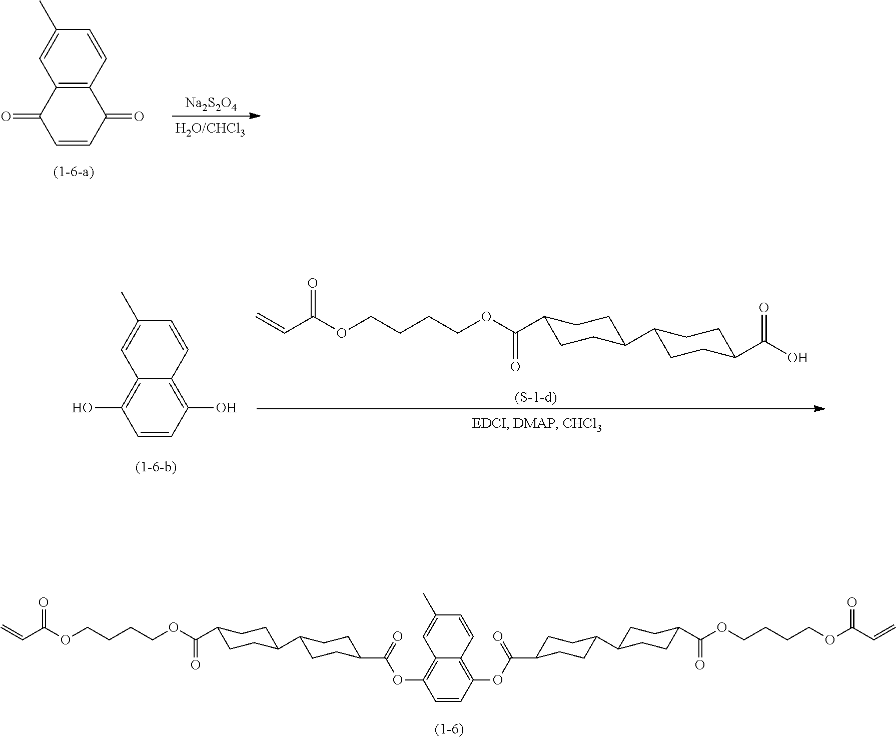 [SMILES: Cc1ccc2c(c1)C(=O)C=CC2=O ; "Na2S2O4" ; Cc1ccc2c(O)ccc(O)c2c1 ; C=CC(=O)OCCCCOC(=O)C1CCC(C2CCC(C(=O)O)CC2)CC1 ; C=CC(=O)OCCCCOC(=O)C1CCC(C2CCC(C(=O)Oc3ccc(OC(=O)C4CCC(C5CCC(C(=O)OCCCCOC(=O)C=C)CC5)CC4)c4cc(C)ccc34)CC2)CC1]

1.7 g (yield 65%) of a compound (1-6) was obtained using 0.3 g (1.72 mmol) of the obtained compound (1-6-b) by the synthesis in the same manner as for the compound (1-2), except that the raw material was changed to 6-methyl-1,4-naphthoquinone (1-6-a).

The ¹H-NMR of the obtained compound (1-6) is shown below.

¹H-NMR (solvent: CDCl₃) δ (ppm): 7.73 (d, 1H), 7.59 (s, 1H), 7.36 (d, 1H), 7.18-7.08 (m, 2H), 6.42 (d, 2H), 6.15 (dd, 2H), 5.86 (d, 2H), 4.20 (t, 4H), 4.13 (t, 4H), 2.71-2.60 (m, 2H), 2.51 (s, 3H), 2.36-2.19 (m, 6H), 2.08-1.52 (m, 241H), 1.49-1.35 (m, 4H), 1.27-0.99 (m, 12H)

### Example 7

[Synthesis of Compound (1-7)]

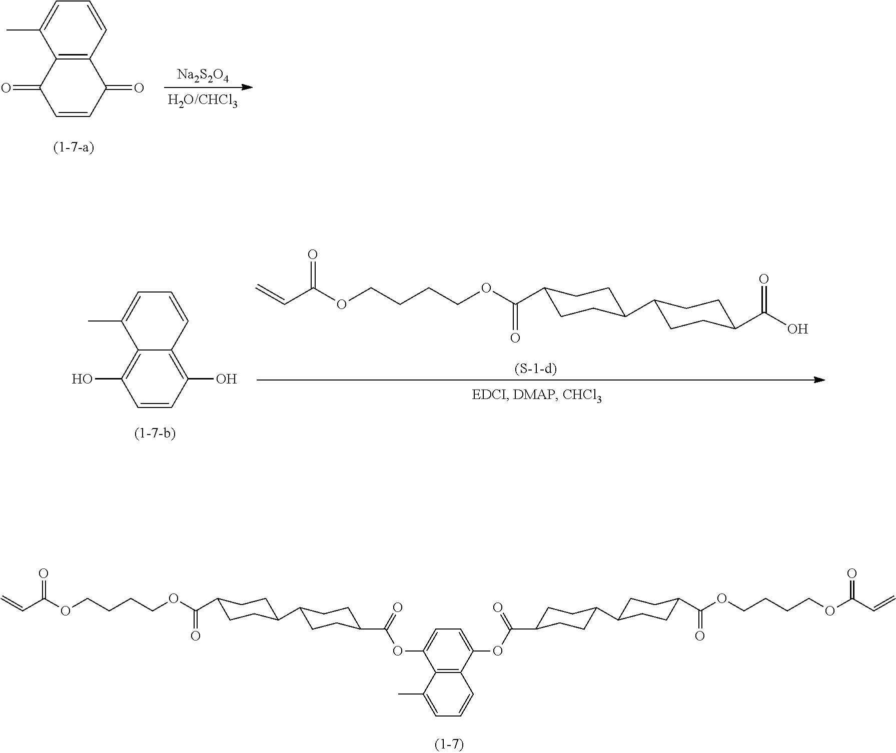

1.5 g (yield 54%) of a compound (1-7) was obtained using 0.5 g (3.12 mmol) of the obtained compound (1-7-b) by the synthesis in the same manner as for the compound (1-2), except that the raw material was changed to 5-methyl-1,4-naphthoquinone (1-7-a).

### Example 8

[Synthesis of Compound (1-8)]

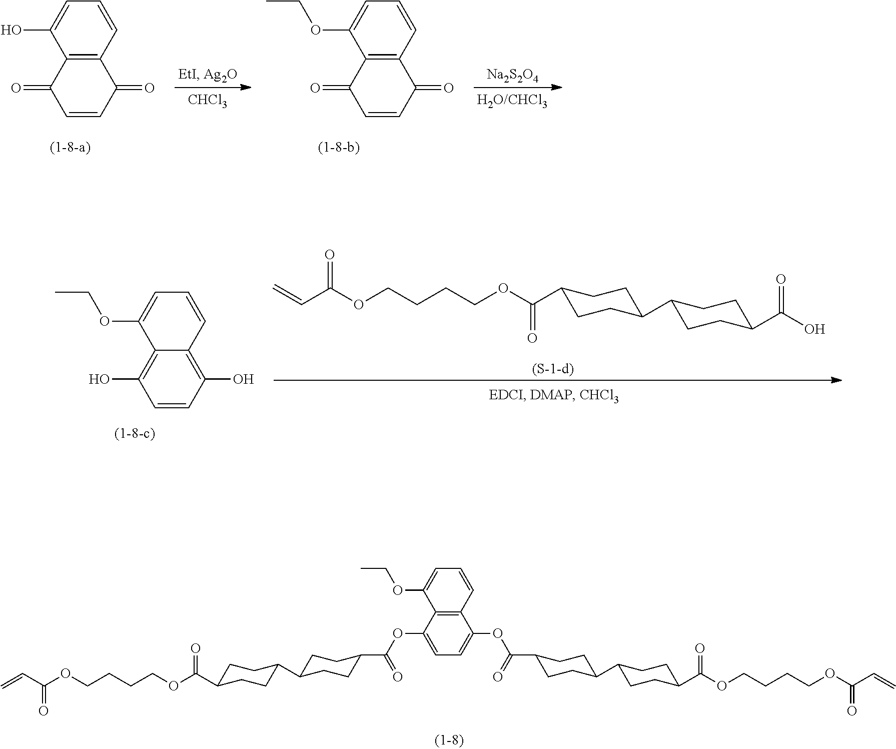

13.8 mL (172 mmol) of ethyl iodide and 5.05 g (21.8 mmol) of silver (I) oxide were added to a solution of 5.0 g (28.7 mmol) of 5-hydroxy-1,4-naphthoquinone (1-8-a) in 200 mL of chloroform, and the mixture was reacted for 15 hours under heating and reflux. 6.9 mL (86.0 mmol) of ethyl iodide and 5.05 g (21.8 mmol) of silver (1) oxide were added thereto twice in total for 8 hours and 13 hours after the start of the reaction. After the reaction, silver oxide was removed by filtration and the solution was concentrated to obtain 5.7 g (yield 98%) of a compound (1-8-b).

Next, 2.75 g (yield 47%) of a compound (1-8-c) was obtained by performing the reaction in the same manner as for the compound (1-2-a), using 5.7 g of the compound (1-8-b).

Lastly, 0.39 g (yield 10%) of a compound (1-8) was obtained by performing the reaction in the same manner as for the compound (1-1), using 0.86 g (4.21 mmol) of the compound (1-8-c).

The ¹H-NMR of the obtained compound (1-8) is shown below.

¹H-NMR (solvent: CDCl₃) δ (ppm): 7.45-7.33 (m, 2H), 7.17 (d, 1H), 6.93 (d, 1H), 6.85 (d, 1H), 6.40 (d, 2H), 6.14 (dd, 2H), 5.83 (d, 2H), 4.21-4.05 (m, 10H), 3.48 (s, 3H), 2.69-2.49 (m, 2H), 2.33-2.12 (m, 6H), 2.09-1.32 (m, 31H), 1.20-0.99 (m, 12H)

### Example 9

[Synthesis of Compound (1-9)]

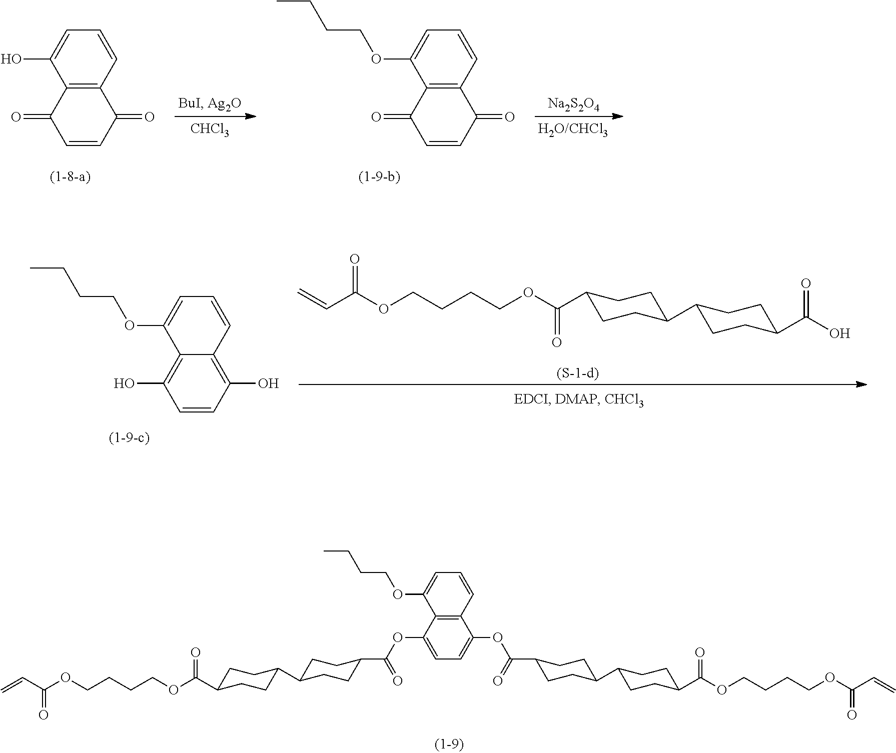

4.4 g (yield 67%) of a compound (1-9-b) was obtained by performing the reaction in the same manner as in the synthesis of the compound (1-8-b), using 5.0 g (28.7 mmol) of 5-hydroxy-1,4-naphthoquinone (1-8-a) and butyl iodide.

Next, 4.25 g (yield 95%) of a compound (1-9-c) was obtained by performing the reaction in the same manner as in the synthesis of the compound (1-2-b), using 4.4 g (19.3 mmol) of the compound (1-9-b).

Lastly, 1.01 g (yield 25%) of a compound (1-9) was obtained by performing the reaction in the same manner as in the synthesis of the compound (1-1), using 0.98 g (4.21 mmol) of the compound (1-9-c).

The ¹H-NMR of the obtained compound (1-9) is shown below.

¹H-NMR (solvent: CDCl₃) δ (ppm): 7.44-7.33 (m, 2H), 7.17 (d, 111), 6.95 (d, 1H), 6.85 (d, 11H), 6.41 (d, 2H), 6.11 (dd, 2H), 5.84 (d, 2H), 4.19 (t, 4H), 4.13-4.02 (m, 611), 2.68-2.46 (m, 2H), 2.34-2.13 (m, 6H), 2.05-1.32 (m, 35H), 1.20-0.99 (m, 12H)

### Example 10

[Synthesis of Compound (1-10)]

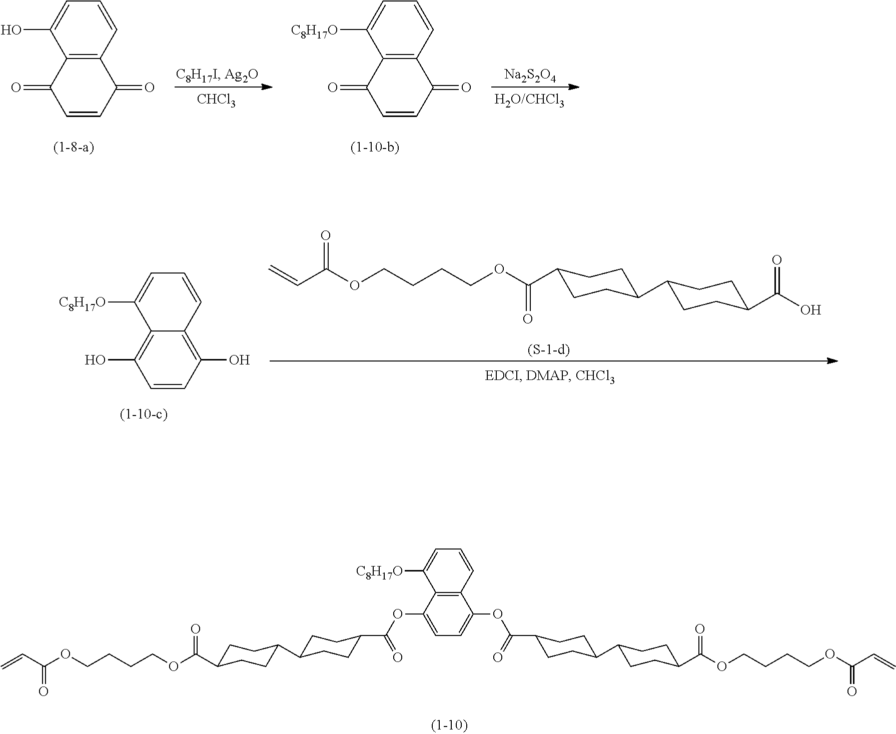

7.55 g (yield 92%) of a compound (1-10-b) was obtained by performing the reaction in the same manner as in the synthesis of the compound (1-8-b), using 5.0 g (28.7 mmol) of 5-hydroxy-1,4-naphthoquinone (1-8-a) and octyl iodide.

Next, 6.53 g (yield 86%) of a compound (1-10-c) was obtained by performing the reaction in the same manner as in the synthesis of the compound (1-2-b), using 7.55 g (19.3 mmol) of compound (1-10-b).

Lastly, 1.60 g (yield 21%) of a compound (1-10) was obtained by performing the reaction in the same manner as in the synthesis of the compound (1-1), using 1.75 g (6.07 mmol) of the compound (1-10-c).

The ¹H-NMR of the obtained compound (1-10) is shown below.

¹H-NMR (solvent: CDCl₃) δ (ppm): 7.42-7.30 (m, 2H), 7.14 (d, 1H), 6.93 (d, 1H), 6.85 (d, 1H), 6.40 (d, 2H), 6.14 (dd, 2H), 5.83 (d, 2H), 4.18 (t, 411), 4.12-4.00 (m, 6H), 2.68-2.44 (m, 2H), 2.34-2.16 (m, 6H), 2.08-0.88 (m, 55H)

### Example 11

[Synthesis of Compound (1-11)]

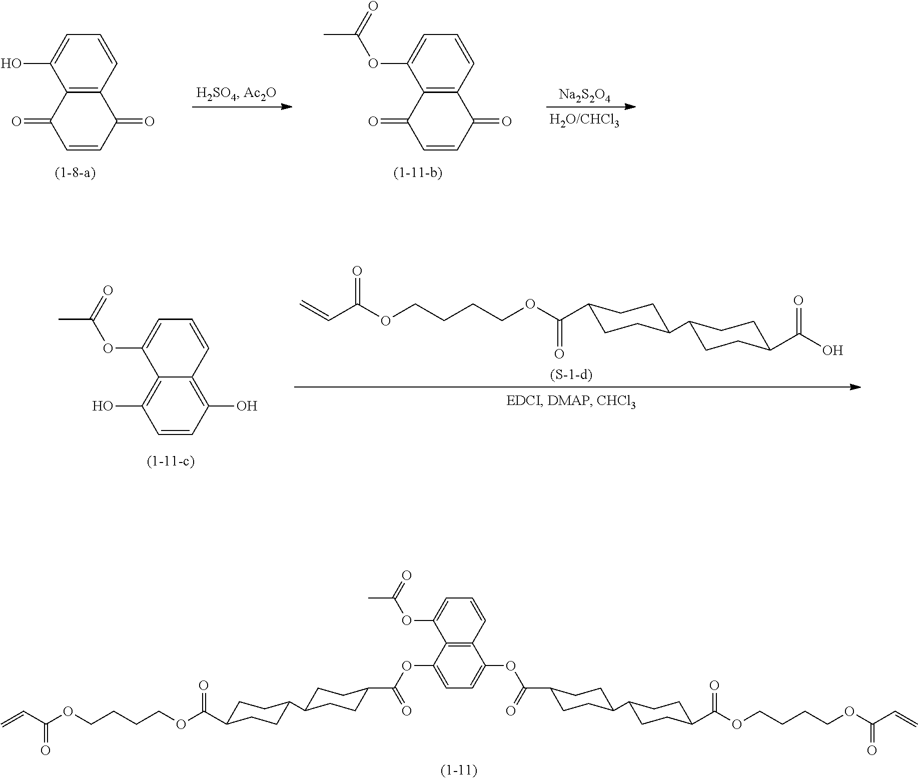

10.3 g (59.1 mmol) of 5-hydroxy-1,4-naphthoquinone (1-8-a) and 60 mL of acetic anhydride were mixed and cooled in an ice bath. 0.6 mL of concentrated sulfuric acid was added dropwise thereto, and the mixture was stirred in an ice bath for 1 hour and at room temperature for 1 hour. The reaction solution was added dropwise to 500 mL of water, the precipitated solid was collected by filtration and washed with water, and the solid was purified with a silica gel column to obtain 9.06 g (yield 71%) of a compound (1-11-b).

Next, 7.35 g (yield 81%) of a compound (1-11-c) was obtained by performing the reaction in the same manner as in the synthesis of the compound (1-2-b), using 9.0 g (41.6 mmol) of the compound (1-11-b).

Lastly, 10.9 g (yield 63%) of a compound (1-11) was obtained by performing the reaction in the same manner as in the synthesis of the compound (1-1), using 4.0 g (18.3 mmol) of the compound (1-11-c).

The ¹H-NMR of the obtained compound (1-11) is shown below.

¹H-NMR (solvent: CDCl₃) δ (ppm): 7.81 (d, 1H), 7.49 (t, 1H), 7.24 (d, 1H), 7.15-7.01 (m, 2H), 6.40 (d, 211), 6.12 (dd, 211), 5.83 (d, 2H), 4.20 (t, 4H), 4.11 (t, 4H), 2.70-2.46 (m, 2H), 2.35 (s, 31), 2.32-2.16 (m, 6H), 2.06-0.98 (m, 40H)

### Example 12

[(Synthesis of Compound (1-12)]

<Synthesis of Carboxylic Acid Derivative (S-2-d)>

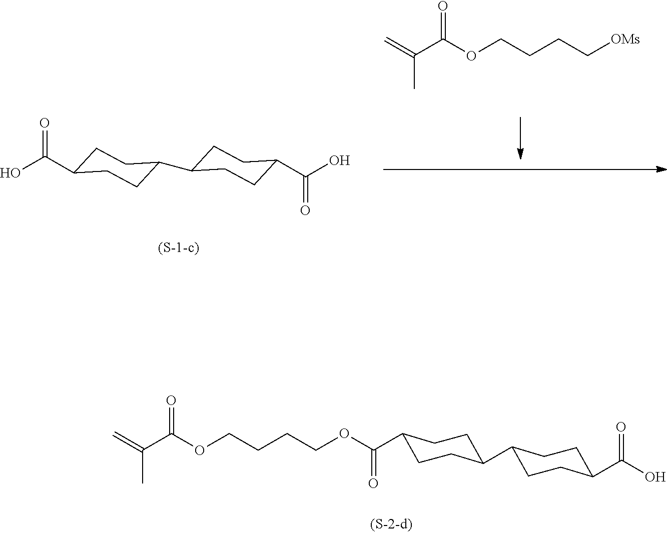 [SMILES: C=C(C)C(=O)OCCCCOS(C)(=O)=O ; O=C(O)C1CCC(C2CCC(C(=O)O)CC2)CC1 ; C=C(C)C(=O)OCCCCOC(=O)C1CCC(C2CCC(C(=O)O)CC2)CC1]

A compound (S-2-d) was synthesized by performing the reaction in the same manner as for the compound (S-1-d), except that 4-methylsulfonyloxybutyl acrylate was changed to 4-methylsulfonyloxybutyl methacrylate.

<Synthesis of Compound (1-12)>

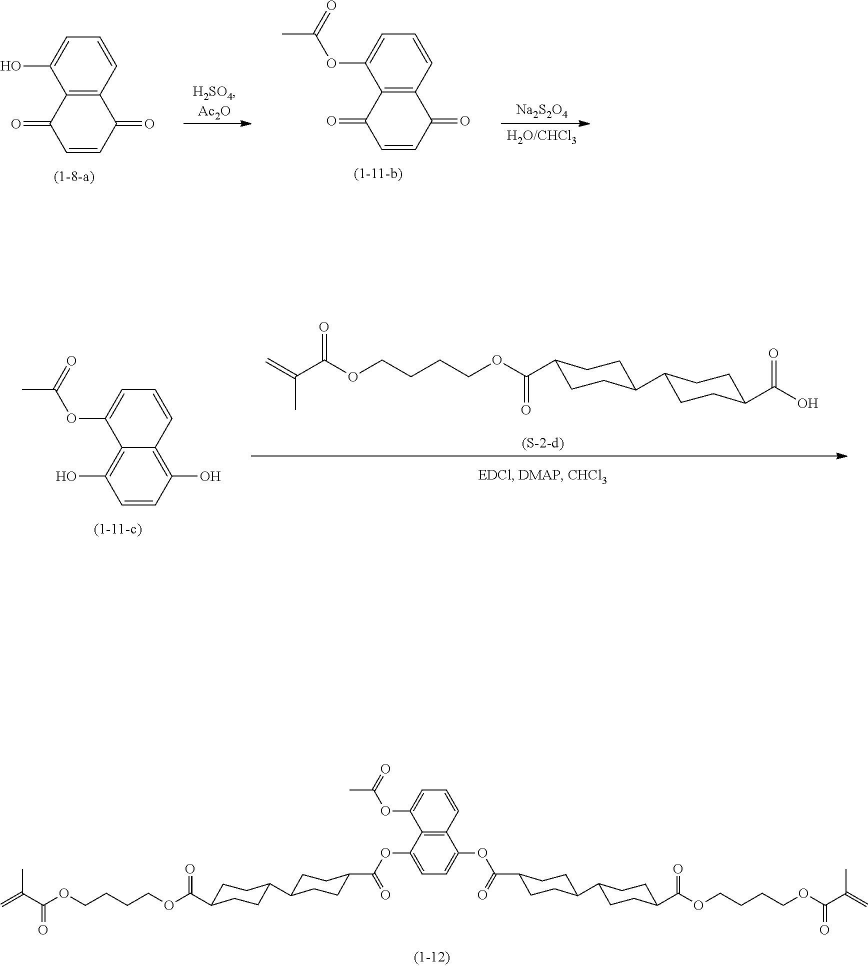

2.55 g (yield 65%) of a compound (1-12) was obtained by performing the reaction in the same manner as in the synthesis of the compound (1-1), using 0.88 g (4.06 mmol) of the compound (1-11-c) and the compound (S-2-d).

The ¹H-NMR of the obtained compound (1-12) is shown below.

¹H-NMR (solvent: CDCl₃) δ (ppm): 7.81 (d, 1H), 7.47 (t, 1H), 7.25 (d, 1H), 7.12-7.06 (m, 2H), 6.10 (s, 2H), 5.58 (s, 2H), 4.19 (t, 4H), 4.10 (t, 4H), 2.72-2.47 (m, 2H), 2.36 (s, 3H), 2.34-2.16 (m, 6H), 2.06-1.32 (m, 28H), 1.22-0.98 (m, 12H)

### Example 13

[Synthesis of Compound (1-13)]

<Synthesis of Carboxylic Acid Derivative (S-3-d)>

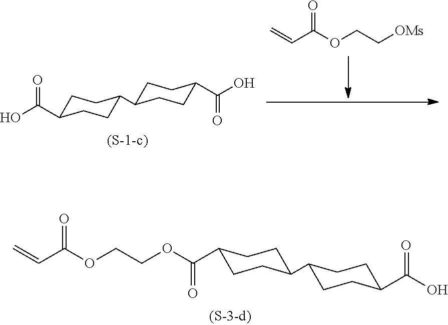 [SMILES: C=CC(=O)OCCOS(C)(=O)=O ; O=C(O)C1CCC(C2CCC(C(=O)O)CC2)CC1 ; C=CC(=O)OCCOC(=O)C1CCC(C2CCC(C(=O)O)CC2)CC1]

A compound (S-3-d) was synthesized by performing the reaction in the same manner as for the compound (S-1-d), except that 4-methylsulfonyloxybutyl acrylate was changed to 4-methylsulfonyloxyethyl acrylate.

<Synthesis of Compound (1-13)

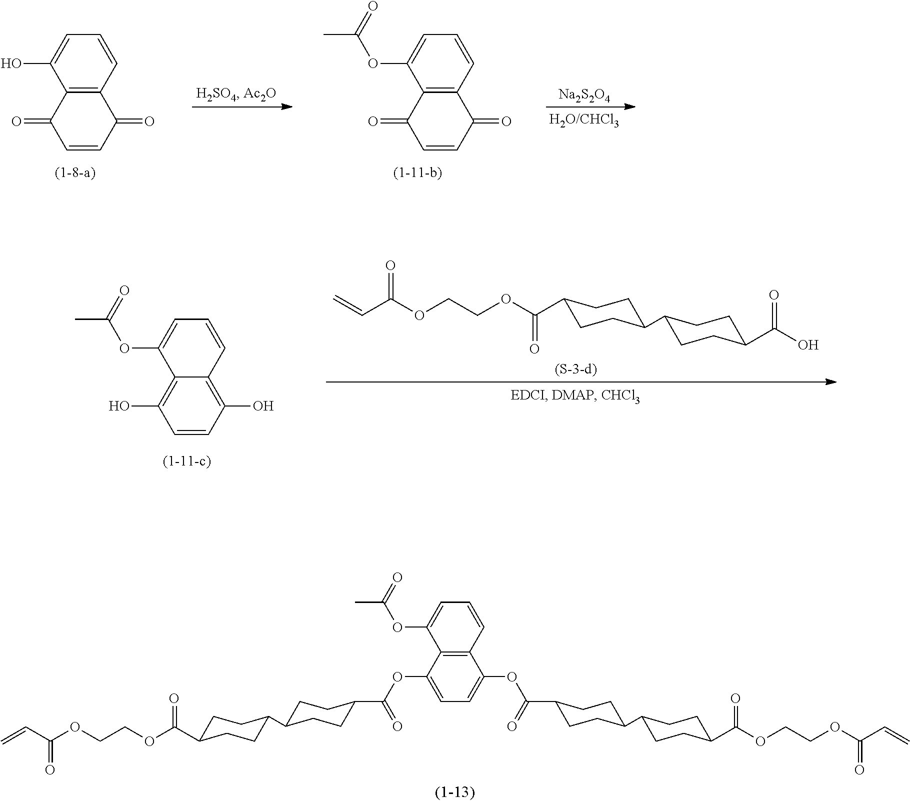

0.97 g (yield 30%) of a compound (1-13) was obtained by performing the reaction in the same manner as in the synthesis of the compound (1-1), using 0.80 g (3.67 mmol) of the compound (1-11-c) and the compound (S-3-d).

The ¹H-NMR of the obtained compound (1-13) is shown below.

¹H-NMR (solvent: CDCl₃) δ (ppm): 7.80 (d, 1H), 7.48 (t, 1H), 7.24 (d, 1H), 7.15-7.03 (m, 211), 6.44 (d, 2H), 6.13 (dd, 2H), 5.88 (d, 2H), 4.41-4.29 (m, 8H), 2.69-2.43 (m, 2H), 2.38 (s, 3H), 2.33-2.29 (m, 6H), 2.07-1.34 (m, 28H), 1.22-0.98 (m, 12H)

### Example 14

[Synthesis of Compound (1-14)]

<Synthesis of Carboxylic Acid Derivative (S-4-d)>

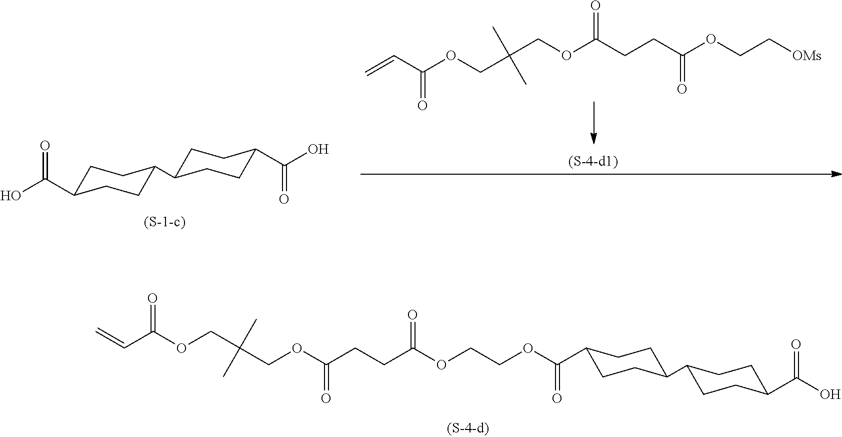 [SMILES: C=CC(=O)OCC(C)(C)COC(=O)CCC(=O)OCCOS(C)(=O)=O ; O=C(O)C1CCC(C2CCC(C(=O)O)CC2)CC1 ; C=CC(=O)OCC(C)(C)COC(=O)CCC(=O)OCCOC(=O)C1CCC(C2CCC(C(=O)O)CC2)CC1]

A compound (S-4-d) was synthesized by performing the reaction in the same manner as for the compound (S-1-d), except that 4-methylsulfonyloxybutyl acrylate was changed to the compound (S-4-d1).

<Synthesis of Compound (1-14)>

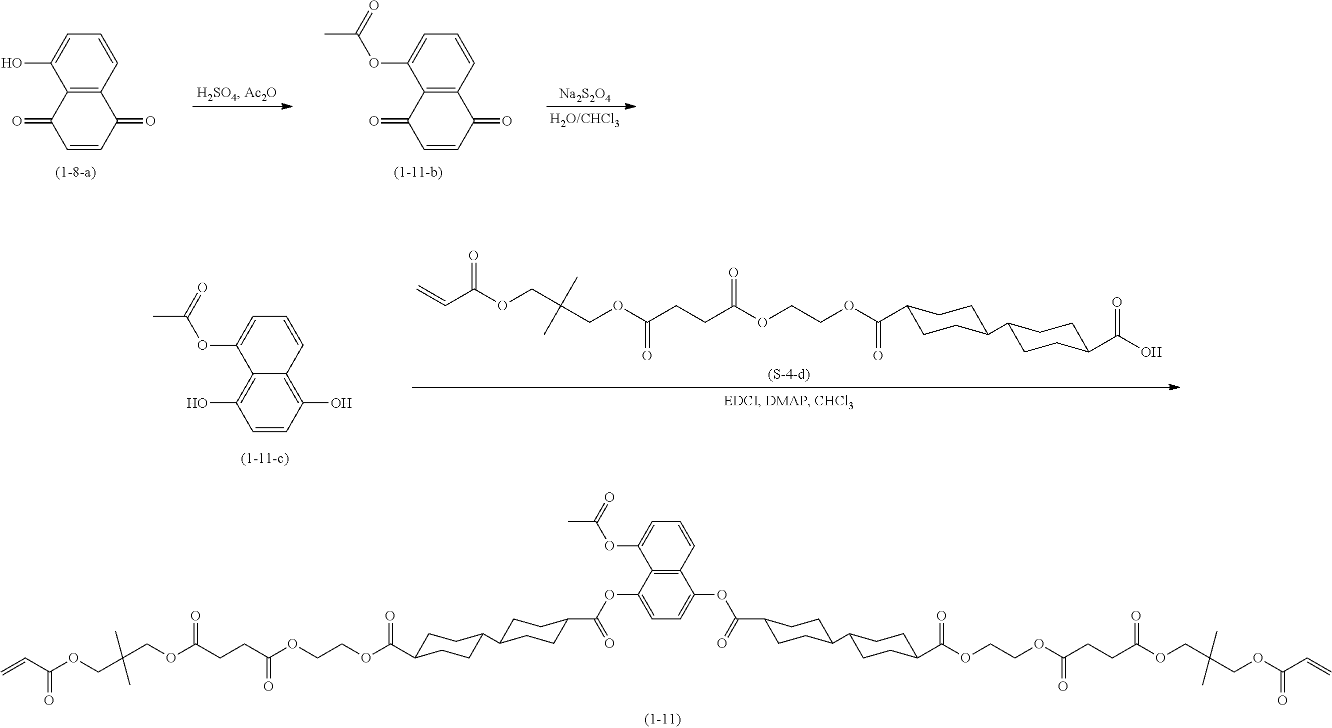

0.93 g (yield 33%) of a compound (1-14) was obtained by performing the reaction in the same manner as in the synthesis of the compound (1-1), using 0.51 g (2.34 mmol) of the compound (1-11-c) and the compound (S-4-d).

The ¹H-NMR of the obtained compound (1-14) is shown below.

¹H-NMR (solvent: CDCl₃) δ (ppm): 7.80 (d, 1H), 7.49 (t, 1H), 7.25 (d, 1H), 7.16-7.04 (m, 2H), 6.42 (d, 2H), 6.19-6.06 (m, 211), 5.89-5.81 (m, 2H), 5.26-5.15 (m, 2H), 4.32-4.10 (m, 12H), 2.71-2.46 (m, 10H), 2.38 (s, 3H), 2.36-2.19 (m, 6H), 2.09-1.25 (m, 28H), 1.24-0.98 (m, 12H)

### Example 15

[Synthesis of Compound (1-15)]

<Synthesis of Carboxylic Acid Derivative (S-5-d)>

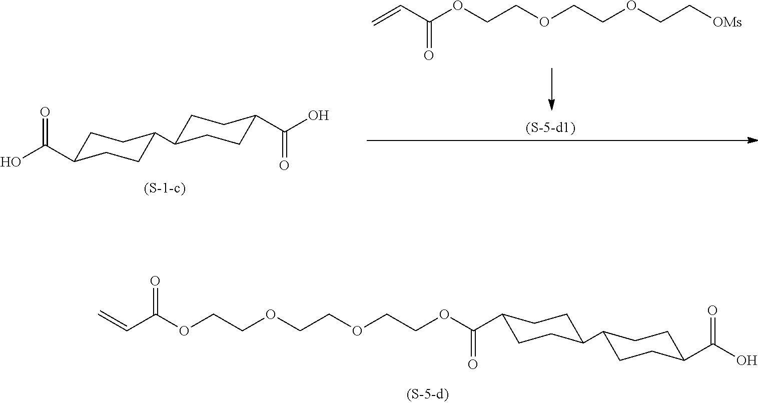 [SMILES: C=CC(=O)OCCOCCOCCOS(C)(=O)=O ; O=C(O)C1CCC(C2CCC(C(=O)O)CC2)CC1 ; C=CC(=O)OCCOCCOCCOC(=O)C1CCC(C2CCC(C(=O)O)CC2)CC1]

A compound (S-5-d) was synthesized by performing the reaction in the same manner as for the compound (S-1-d), except that 4-methylsulfonyloxybutyl acrylate was changed to the compound (S-5-d1).

<Synthesis of Compound (1-15)>

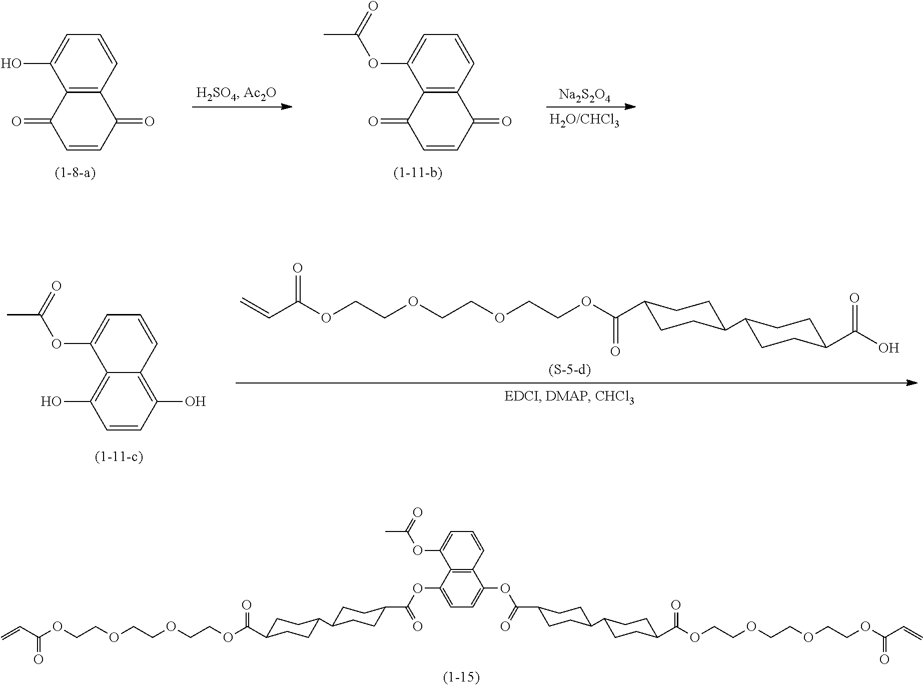

0.98 g (yield 39%) of a compound (1-15) was obtained by performing the reaction in the same manner as in the synthesis of the compound (1-1), using 0.51 g (2.34 mmol) of the compound (1-11-c) and the compound (S-5-d).

The ¹H-NMR of the obtained compound (1-15) is shown below.

¹H-NMR (solvent: CDCl₃) δ (ppm): 7.80 (d, 1H), 7.49 (t, 1H), 7.26 (d, 1H), 7.16-7.05 (m, 2H), 6.45 (d, 2H), 6.18 (dd, 2H), 5.87 (d, 2H), 4.37-4.30 (m, 4H), 4.21 (t, 4H), 3.79-3.61 (m, 16H), 2.70-2.46 (m, 2H), 2.38 (s, 3H), 2.32-2.16 (m, 6H), 2.09-1.32 (m, 28H), 1.21-0.96 (m, 12H)

### Example 16

[Synthesis of Compound (1-16)]

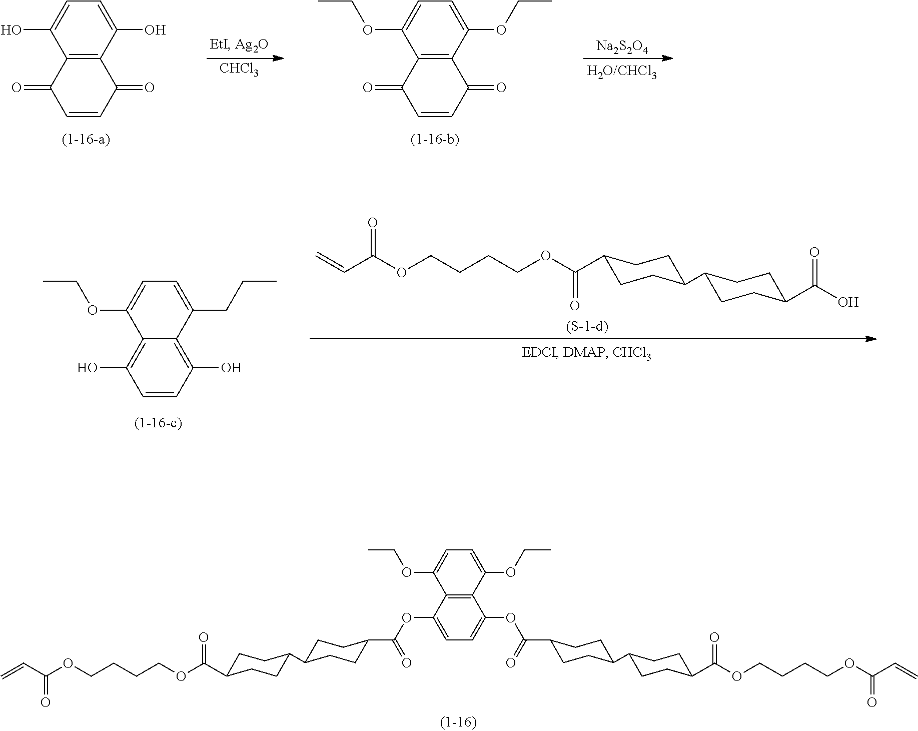

6.4 mL (78.9 mmol) of ethyl iodide and 4.65 g (20.0 mmol) of silver (I) oxide were added to a solution of 5.0 g (26.3 mmol) of 5,8-dihydroxy-1,4-naphthoquinone (1-16-a) in 250 mL of chloroform, and the mixture was reacted for 5 days under heating and reflux. 6.4 mL (78.9 mmol) of ethyl iodide and 4.65 g (20.0 mmol) of silver (I) oxide were added four times every 24 hours during the reaction. After the reaction, silver oxide was removed by filtration, and the solution was concentrated and then purified with a silica gel column. 1.4 g (yield 21%) of a compound (1-16-b) was obtained.

Next, 0.86 g (yield 61%) of a compound (1-16-c) was obtained by performing the reaction in the same manner as for the compound (1-2-a), using 1.4 g of the compound (1-16-b).

Lastly, 0.33 g (yield 10%) of a compound (1-16) was obtained by performing the reaction in the same manner as for the compound (1-1), using 0.86 g (3.46 mmol) of the compound (1-16-c).

The ¹H-NMR of the obtained compound (1-16) is shown below.

¹H-NMR (solvent: CDCl₃) δ (ppm): 6.93 (s, 2H), 6.75 (s, 2H), 6.39 (d, 2H), 6.10 (dd, 2H), 5.81 (d, 2H), 4.20 (t, 4H), 4.14-4.03 (m, 8H), 2.60-2.56 (m, 2H), 2.31-2.15 (m, 6H), 2.05-1.31 (m, 34H), 1.19-0.98 (m, 12H)

### Example 17

[Synthesis of Compound (1-17)]

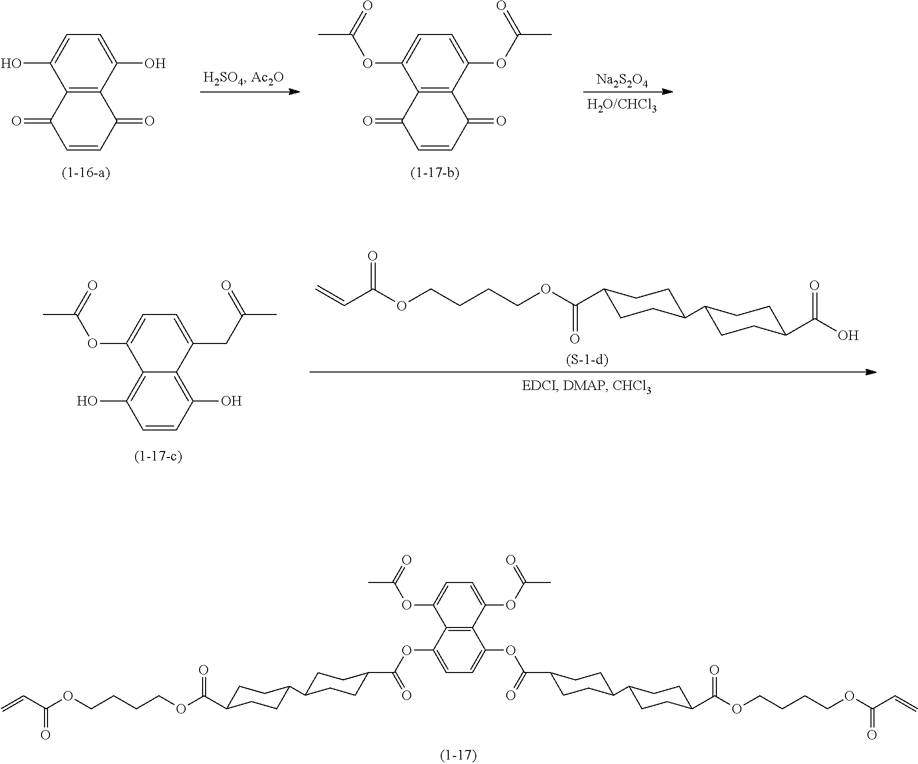

1.5 g (7.89 mmol) of 5,8-dihydroxy-1,4-naphthoquinone (1-16-a) and 20 mL of acetic anhydride were mixed and cooled in an ice bath. 0.5 mL of concentrated sulfuric acid was added dropwise thereto, and the mixture was stirred in an ice bath for 1 hour and at room temperature for 1 hour. A reaction solution was added dropwise to 200 mL of water, the precipitated solid was collected by filtration and washed with water, and the solid was purified with a silica gel column to obtain 1.67 g (yield 77%) of a compound (1-17-b).

Next, 1.35 g (yield 80%) of a compound (1-17-c) was obtained by performing the reaction in the same manner as in the synthesis of the compound (1-2-b), using 1.67 g (6.09 mmol) of the compound (1-17-b).

Lastly, 1.38 g (yield 29%) of a compound (1-17) was obtained by performing the reaction in the same manner as in the synthesis of the compound (1-1), using 1.3 g (4.71 mmol) of the compound (1-17-c).

The ¹H-NMR of the obtained compound (1-17) is shown below.

¹H-NMR (solvent: CDCl₃) δ (ppm): 7.13-7.03 (m, 4H), 6.41 (d, 2H), 6.13 (dd, 2H), 5.82 (d, 2H), 4.19 (t, 4H), 4.09 (t, 4H), 2.58-2.44 (m, 2H), 2.35 (s, 6H), 2.28-2.16 (m, 6H), 2.06-1.32 (m, 28H), 1.19-0.97 (m, 12H)

### Example 18

[Synthesis of Compound (2-1)]

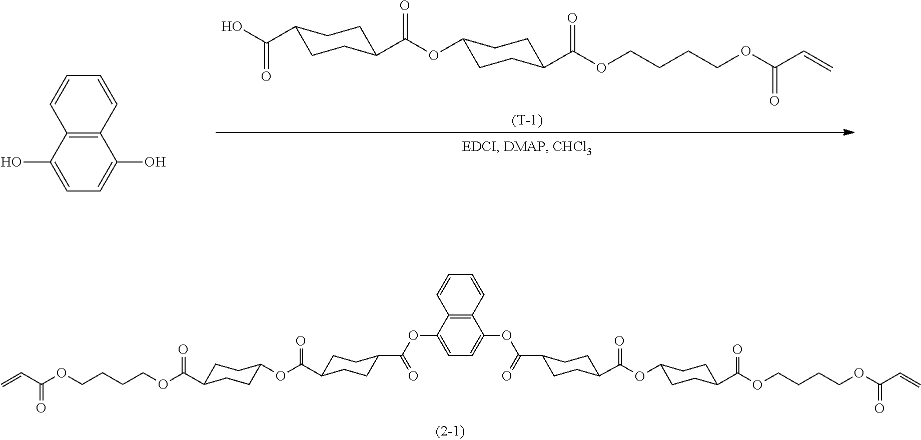

1.3 g (yield 43%) of a compound (2-1) was obtained by performing the reaction in the same manner as for the compound (1-1), using 0.5 g (3.12 mmol) of 1,4-dihydroxynaphthalene and 3.31 g (7.80 mmol) of the compound (T-1).

The ¹H-NMR of the obtained compound (2-1) is shown below.

¹H-NMR (solvent: CDCl₃) δ (ppm): 7.82 (d, 21H), 7.53 (d, 2H), 7.22 (s, 2H), 6.43 (d, 2H), 6.12 (dd, 2H), 5.88 (d, 211), 5.80-5.68 (m, 21H), 4.20 (t, 4H), 4.12 (t, 41H), 2.80-2.69 (m, 2H), 2.42.2.23 (m, 81H), 2.16 (d, 4H), 2.08 (d, 8H), 1.80-1.33 (m, 24H)

### Example 19

[Synthesis of Compound (2-2)]

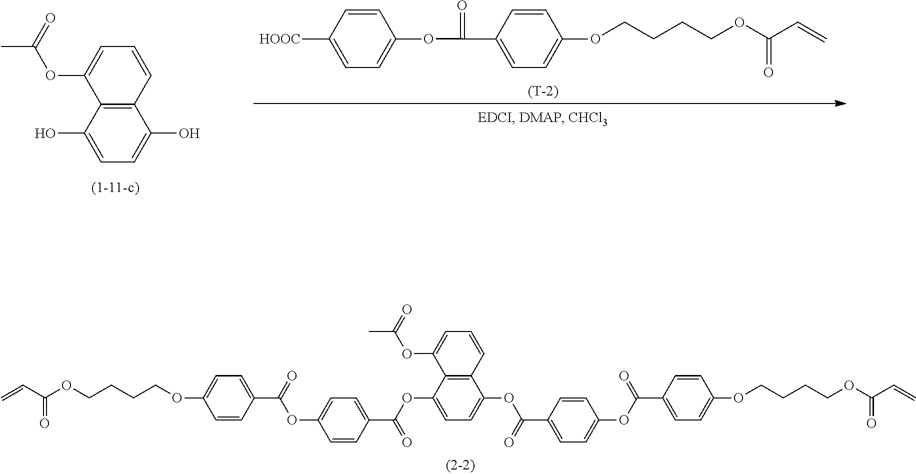

1.2 g (yield 53%) of a compound (2-2) was obtained by performing the reaction in the same manner as in the compound (1-1), using 0.5 g (2.29 mmol) of 5-acetoxy-1,4-dihydroxynaphthalene (A-11-c) and 2.2 g (5.73 mmol) of the compound (T-2).

The ¹H-NMR of the obtained compound (2-2) is shown below.

¹H-NMR (solvent: CDCl₃) δ (ppm): 8.44-8.37 (m, 411), 8.22-8.14 (m, 4H), 7.94 (t, 1H), 7.60-7.49 (m, 6H), 7.30 (d, 111), 7.19 (d, 111), 7.01 (d, 4H), 6.43 (d, 2H), 6.15 (Dd, 211), 5.86 (d, 211), 4.29 (t, 4H), 4.11 (t, 4H), 2.01-1.88 (m, 8H), 1.82 (s, 3H)

### Example 20

[Synthesis of Compound (2-3)]

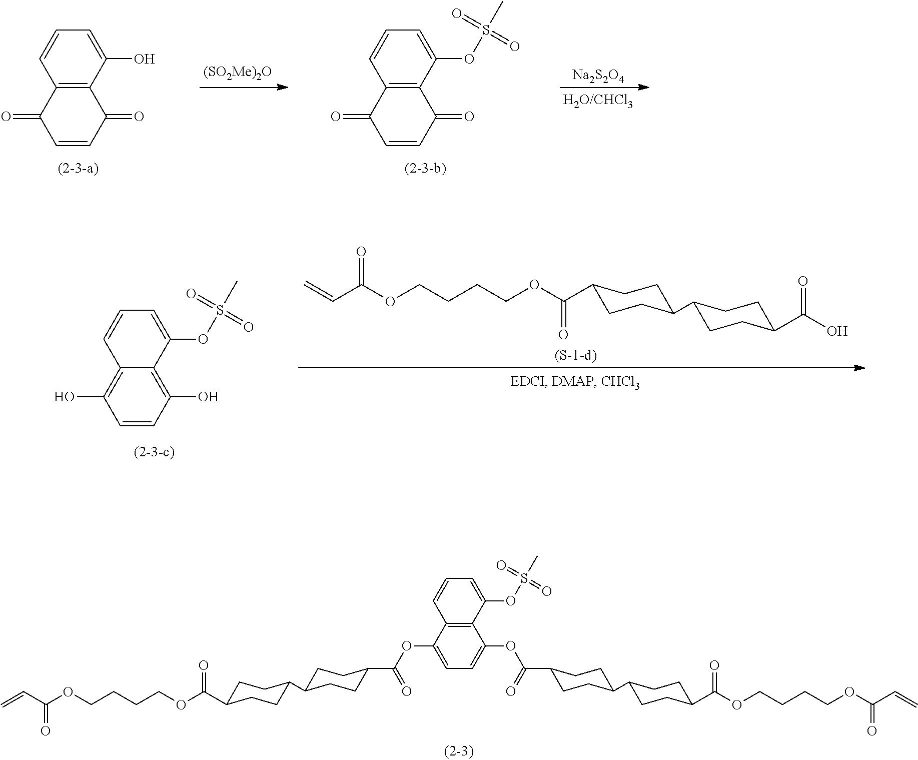

5.00 g (28.7 mmol) of 5-hydroxy-1,4-naphthoquinone was dissolved in 10 mL of dichloromethane, and 7.5 g (43.1 mmol) of methylsulfonylmethanesulfonate was added dropwise thereto at room temperature. Next, 0.35 g (2.87 mmol) of 4-dimethylaminopyridine and 2.50 g (31.6 mmol) of pyridine were added thereto under ice-cooling, and the mixture was stirred at 40° C. for 1 hour. 100 mL of water and 200 mL of ethyl acetate were added thereto to perform a liquid separation operation. 100 mL of saturated aqueous sodium bicarbonate was added to the organic layer to perform a liquid separation operation again. The solvent of the organic layer was evaporated and the residue was purified by silica gel column chromatography to obtain 1.30 g (yield 18%) of an ocher solid (2-3-b). Next, 1.06 g (yield 81%) of a compound (2-3-c) was obtained by performing the reaction in the same manner as in the synthesis of the compound (1-2-b), using 1.30 g (5.15 mmol) of the compound (2-3-b). Lastly, 2.31 g (yield 57%) of a compound (2-3) was obtained by performing the reaction in the same manner as in the synthesis of the compound (1-1), using 1.06 g (4.17 mmol) of the compound (2-3-c).

The ¹H-NMR of the obtained compound (2-3) is shown below.

¹H-NMR (solvent: CDCl₃) δ (ppm): 7.88 (dd, 1H), 7.63 (dd, 1H), 7.52 (t, 1H), 7.29 (d, 1H), 7.16 (d, 1H), 6.42 (dd, 2H), 6.18-6.08 (m, 2H), 5.84 (dd, 2H), 4.15 (d, 8H), 3.01 (s, 3H), 2.82-2.62 (m, 2H), 2.37-2.17 (m, 6H), 2.09-1.33 (m, 28H), 1.25-0.98 (m, 12H)

### Example 21

[Synthesis of Compound (2-4)]

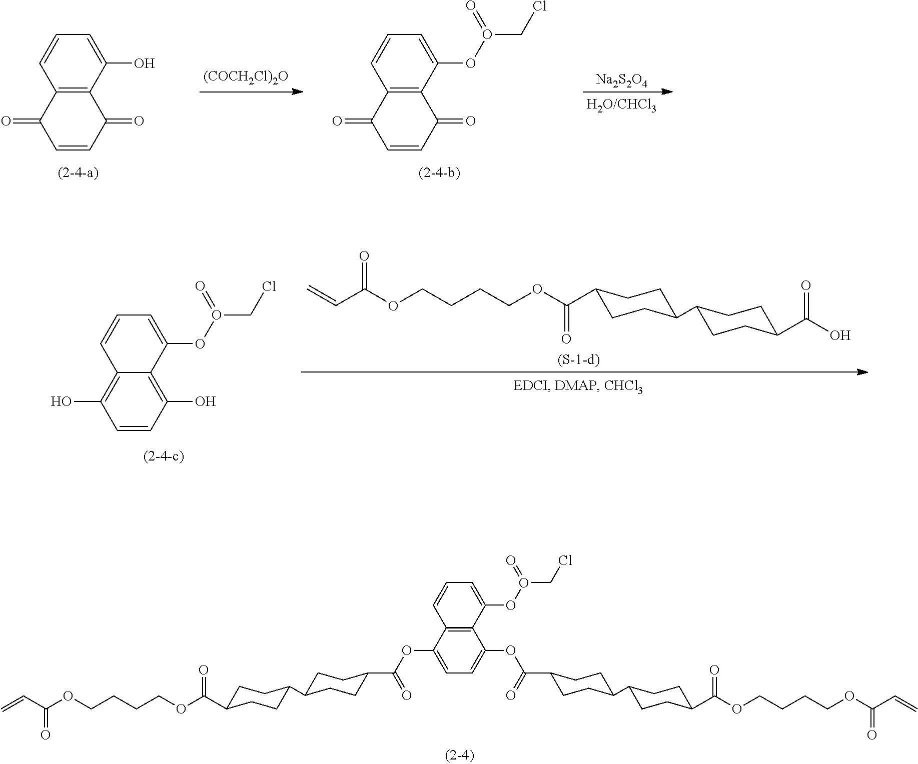

5.00 g (28.7 mmol) of 5-hydroxy-1,4-naphthoquinone was dissolved in 50 mL of dichloromethane, 7.36 g (43.1 mmol) of 2-chloroacetyl-2-chloroacetone, 0.35 g of 4-dimethylaminnpyridine. (2.87 mmol) and 2.50 g (31.6 mmol) of pyridine were added thereto, and the mixture was stirred at room temperature for 1 hour. 200 mL of ethyl acetate and 100 mL of water were added thereto to perform a liquid separation operation. 100 mL of saturated aqueous sodium bicarbonate was added to the organic layer to perform a liquid separation operation again. After dispersion washing with chloroform and ethyl acetate, the mixture was filtered to obtain 2.29 g (yield 32%) of an ocher solid (2-4-a). Next, 1.80 g (yield 81%) a of a compound (2-4-c) was obtained by performing the reaction in the same manner as in the synthesis of the compound (1-2-b), using 2.20 g (8.78 mmol) of the compound (24-b). Lastly, 0.44 g (yield 13%) of a compound (2-4) was obtained by performing the reaction in the same manner as in the synthesis of the compound (1-1), using 0.90 g (3.56 mmol) of the compound (2-4-c).

The ¹H-NMR of the obtained compound (2-4) is shown below.

¹H-NMR (solvent: CDCl₃) δ (ppm): 7.82 (d, 1H), 7.51 (t, 1H), 7.25 (d, 1H), 7.20-7.08 (m, 2H), 6.42 (dd, 211), 6.17-6.08 (m, 2H), 5.84 (dd, 2H), 4.37 (s, 2H), 4.15 (dt, 8H), 2.71-2.44 (m, 2H), 2.33-2.15 (m, 6H), 2.08-1.34 (m, 28H), 1.23-0.98 (m, 12H)

### Comparative Example 1

A comparative compound 1 represented by the following formula was synthesized according to the description in JP2013-164520A.

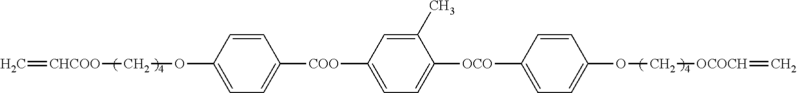

### Comparative Example 2

A comparative compound 2 represented by the following formula, in which a ring structure was further introduced into the above-mentioned comparative compound 1, was synthesized.

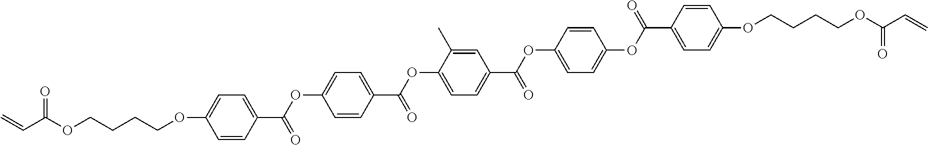 [SMILES: C=CC(=O)OCCCCOc1ccc(C(=O)Oc2ccc(OC(=O)c3ccc(OC(=O)c4ccc(OC(=O)c5ccc(OCCCCOC(=O)C=C)cc5)cc4)c(C)c3)cc2)cc1]

[Evaluation 1]

For each of the compounds synthesized in Examples and Comparative Examples, the phase transition temperature, the precipitation suppression, and the solubility were evaluated by methods shown below.

(1) Phase Transition Temperature

Two polarizers of an optical microscope (ECLIPSE E600 POL, manufactured by Nikon Corporation) were arranged so that they were orthogonal to each other, and a sample stand was set between the two polarizers.

Then, a small amount of each synthesized compound was placed on a slide glass, and the slide glass was set on a hot stage placed on a sample stand. While observing the state of the sample with a microscope, the temperature of the hot stage was raised at 5° C./min, and the type of liquid crystal phase and the temperature at which phase transition occurred were recorded from the state of the sample.

In addition, for the recrystallization temperature, the temperature was elevated up to an isotropic phase and then lowered at 5° C./min, and a temperature at which crystals were precipitated was recorded.

The results are shown in Table 3 below. Further, in Table 3 below, Cr represents a crystal, SA represents a smectic phase, N represents a nematic phase, and Is represents an isotropic phase. In addition, in Example 1 (Compound 1-1) in Table 3 below, “230 or more” in the notation “Cr 185 SA 220 N 230 or more Is” means that “a polymerization proceeds at 230° C. or higher, and thus, a temperature during a phase transition to an isotropic phase cannot be measured”.

(2) Precipitation Suppression

Based on the results of the recrystallization temperature measured by the above-mentioned method for evaluating the phase transition temperature, the evaluation was made according to the following standard. The results are shown in Table 3 below.

A: Lower than 50° C.

B: 50° C. or higher and 100° C. or lower

C: Higher than 100° C. and 150° C. or lower

D: Higher than 150° C. and 180° C. or lower

(3) Solubility

Cyclopentanone was added to 25 mg of each synthesized compound so that the concentration of each compound was 40% by mass, and the mixture was heated and stirred at 50° C. for 1 minute.

Then, the mixture was left to stand at 20° C. for 10 minutes, and in a case where there is no undissolved residue or precipitation, it is determined that the mixture can be dissolved at 40% by mass.

In a case where there was an undissolved residue or precipitation, cyclopentanone was further added to the mixture to reduce the concentration by 5% by mass, an operation of heating and stirring at 50° C. for 1 minute and then leaving the mixture stand at 20° C. for 10 minutes was repeatedly performed until there was no undissolved residue or precipitation, thereby confirming the solubility.

The concentration values indicating the solubility are shown in Table 3. Further, in Table 3 below, “>40” indicates that the substance is dissolved at a concentration of 40% by mass, so that it can also be dissolved even at a concentration of 40% by mass or more, and “<5” indicates that the substance is dissolved at 5% by mass, and the undissolved residues and precipitation are observed.

TABLE 3

| | | Evaluation | | |
|---|---|---|---|---|
| | | | Precipitation | |
| Table 3 | Compound | Phase transition temperature | suppression | Solubility |
| Example 1 | 1-1 | Cr 185 SA 220 N 230 or more Is recrystal | D | 20 |
| | | 174 | | |
| Example 2 | 1-2 | Cr 133 SA 178 N 255 or more Is recrystal | C | 25 |
| | | 123 | | |
| Example 3 | 1-3 | Cr 135-140 N 247 Is recrystal 115 | C | 20 |
| Example 4 | 1-4 | Cr 84 N 208 Is recrystal 50 | B | >40 |
| Example 5 | 1-5 | Cr 105 N 230 Is recrystal 70 | B | >40 |
| Example 6 | 1-6 | Cr 117 SA 162 N 255 Is recrystal 110 | C | 35 |
| Example 7 | 1-7 | Cr 126 SA 201 Iso 250 or more Is | C | 25 |
| Example 8 | 1-8 | Cr 87 SA 221 N 265 or more Is recrystal 65 | B | 20 |
| Example 9 | 1-9 | Cr 79 SA 155 N 228 Is recrystal 49 | A | 20 |
| Example 10 | 1-10 | Cr 110 SA 166.5 N 219 Is recrystal 70 | B | 30 |
| Example 11 | 1-11 | Cr 82 SA 197 N 255 Is | A | 35 |
| | | recrystal room temperature or lower | | |
| Example 12 | 1-12 | Cr 105 SA 225-230 N 260 Is | A | 35 |
| | | recrystal room temperature or lower | | |
| Example 13 | 1-13 | Cr 70-75 SA 185 N 280 Is | A | 30 |
| | | recrystal room temperature or lower | | |
| Example 14 | 1-14 | Room temperature SA 116-120 N 125-133 Is | A | >40 |
| | | recrystal room temperature or lower | | |
| Example 15 | 1-15 | Cr 55 SA 145-158 N 190-220 Is | A | >40 |
| | | recrystal room temperature or lower | | |
| Example 16 | 1-16 | Cr 62 SA 189 N 236 Is recrystal 33 | A | >40 |
| Example 17 | 1-17 | Cr 118-125 SA 132 N 240 Is recrystal 116 | C | 20 |
| Example 18 | 2-1 | Cr 148 N 233 Is recrystal 140 | C | 10 |
| Example 19 | 2-2 | Cr 142 N-270 or more Is recrystal 110 | C | 15 |
| Example 20 | 2-3 | Cr 128 (SA 105) N 236 Is | A | 25 |
| | | recrystal room temperature or lower | | |
| Example 21 | 2-4 | Cr 92 SA 193 N 256 Is | A | 20 |
| | | recrystal room temperature or lower | | |
| Comparative | Comparative | Cr 80 N 124 Is recrystal <50 | A | >40 |
| Example 1 | compound 1 | | | |
| Comparative | Comparative | Cr 133 N 250 or more Is recrystal 102 | C | <5 |
| Example 2 | compound 2 | | | |

From the results shown in Table 3 above, it was found that a comparative compound 1 had a narrow temperature range exhibiting liquid crystallinity, but had good precipitation suppression and solubility (Comparative Example 1).

In addition, it was found that in a case where a ring structure was further introduced into the comparative compound 1, the temperature range exhibiting liquid crystallinity was widened, but the precipitation suppression and the solubility were deteriorated (Comparative Example 2).

In contrast, it was found that a predetermined compound having a naphthalene skeleton having a side chain structure at the 1,4 position in the center (core) of the molecule serves as a compound having a wide temperature range exhibiting liquid crystallinity and excellent precipitation suppression and solubility (Examples 1 to 21).

### Examples 22 to 30

<Formation of Alignment Film P-3>

Using a glass plate as a temporary support for formation, the following coating liquid for forming an alignment film P-3 was applied onto a glass substrate using a #18 bar coater, and the glass substrate was dried with hot air at 100° C. for 120 seconds and then subjected to a rubbing treatment to form an alignment film P-3.

(Coating Liquid for Forming Alignment Film P-3)

| | |
|---|---|
| Polyvinyl alcohol (PVA 203, manufactured | 2.0 parts by mass |
| by Kuraray C., Ltd.) | |
| Water | 98.0 parts by mass |

<Formation of Polymerizable Composition Layer>

The following polymerizable composition was applied onto the alignment film P-3 by a spin coating method.

(Polymerizable Composition)

| | |
|---|---|
| The following polymerizable liquid crystal compound L-1 | 32.00 parts by mass |
| The following polymerizable liquid crystal compound L-2 | 48.00 parts by mass |
| Compounds described in Table 4 below | 20.00 parts by mass |
| The following polymerization initiator PI-1 | 0.50 parts by mass |
| The leveling agent T-1 | 0.20 parts by mass |
| Cyclopentanone | 235.00 parts by mass |

Polymerizable liquid crystal compound L-1

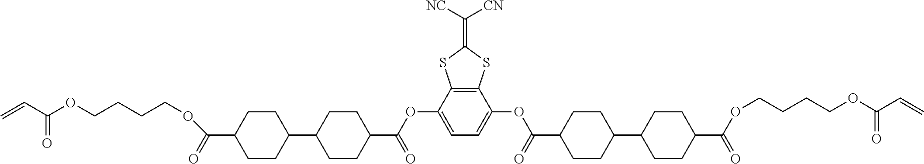 [SMILES: C=CC(=O)OCCCCOC(=O)C1CCC(C2CCC(C(=O)Oc3ccc(OC(=O)C4CCC(C5CCC(C(=O)OCCCCOC(=O)C=C)CC5)CC4)c4c3SC(=C(C#N)C#N)S4)CC2)CC1]

Polymerizable liquid crystal compound L-2

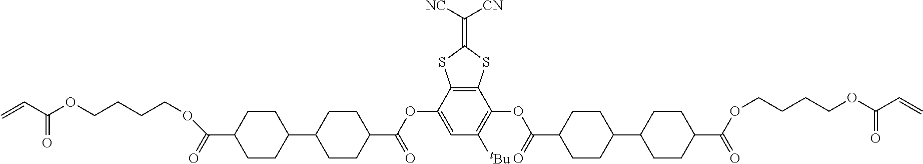 [SMILES: C=CC(=O)OCCCCOC(=O)C1CCC(C2CCC(C(=O)Oc3cc(C(C)(C)C)c(OC(=O)C4CCC(C5CCC(C(=O)OCCCCOC(=O)C=C)CC5)CC4)c4c3SC(=C(C#N)C#N)S4)CC2)CC1]

Polymerization initiator PI-1

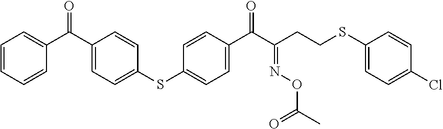 [SMILES: CC(=O)O/N=C(\CCSc1ccc(Cl)cc1)C(=O)c1ccc(Sc2ccc(C(=O)c3ccccc3)cc2)cc1]

Leveling agent T-1

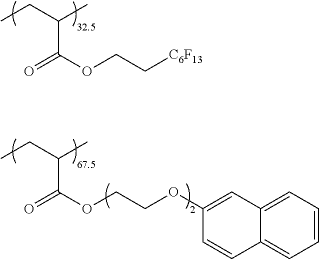

### Comparative Example 3

A polymerizable composition was applied onto the alignment film P-3 by a spin coating method by the same method as in Example 22, except that the compound (1-3) was not blended.

[Evaluation 2]

The phase transition temperature of the polymerizable composition applied on the alignment film P-3 in Examples 22 to 30 and Comparative Example 3 was measured by a method shown below.

Two polarizers of an optical microscope (ECLIPSE E600 POL, manufactured by Nikon Corporation) were arranged so that they were orthogonal to each other, and a sample stand was set between the two polarizers.

Then, a small amount of the prepared polymerizable composition was placed on a slide glass, and the slide glass was set on a hot stage placed on a sample stand. While observing the state of the sample with a microscope, a temperature range of the smectic phase (Sm) was calculated by measuring the upper limit temperature and the crystallization temperature of the smectic phase (Sm) while elevating the temperature to a nematic phase and then lowering the temperature at 10° C./min. The results are shown in Table 4 below.

TABLE 4

| | | | Evaluation | | |
|---|---|---|---|---|---|
| | | | Sm upper limit | Crystallization | Sm temperature |
| | | Polymerizable liquid | temperature | temperature | range |
| Table 4 | Compound | crystal compund | (° C.) | (° C.) | (° C.) |
| Example 22 | 1-3 | L-1/L-2 | 121 | 60 | 61 |
| Example 23 | 1-6 | L-1/L-2 | 142 | <50 | >92 |
| Example 24 | 1-8 | L-1/L-2 | 149 | <60 | >89 |
| Example 25 | 1-9 | L-1/L-2 | 126 | <45 | >81 |
| Example 26 | 1-10 | L-1/L-2 | 138 | <50 | >88 |
| Example 27 | 1-11 | L-1/L-2 | 140 | <50 | >90 |
| Example 28 | 1-12 | L-1/L-2 | 150 | <50 | >100 |
| Example 29 | 1-13 | L-1/L-2 | 129 | <50 | >79 |
| Example 30 | 1-16 | L-1/L-2 | 138 | 54 | 84 |
| Comparative | — | L-1/L-2 | 100 | 84 | 16 |
| Example 3 | | | | | |

From the results shown in Table 4 above, it was found that in a case where the compound (1) was blended into the polymerizable liquid crystal compound, the temperature range exhibiting liquid crystallinity (in particular, smectic liquid crystallinity) was widened (Examples 22 to 30).

### Examples 31 to 33

[Manufacture of Cellulose Acylate Film 1]

<Manufacture of Core Layer Cellulose Acylate Dope>

The following composition was put into a mixing tank and stirred to dissolve the respective components to prepare a cellulose acetate solution for use as a core layer cellulose acylate dope.

Core layer cellulose acylate dope

| | |
|---|---|
| Cellulose acetate having a degree of acetyl substitution of 2.88 | 100 parts by mass |
| Polyester compound B described in Examples of JP2015-227955A | 12 parts by mass |
| The following compound F | 2 parts by mass |
| Methylene chloride (first solvent) | 500 parts by mass |
| Methanol (second solvent) | 75 parts by mass |

Compound F

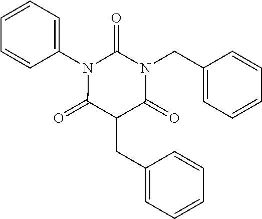 [SMILES: O=C1C(Cc2ccccc2)C(=O)N(c2ccccc2)C(=O)N1Cc1ccccc1]

<Manufacture of Outer Layer Cellulose Acylate Dope>

10 parts by mass of the following matting agent solution was added to 90 parts by mass of the core layer cellulose acylate dope to prepare a cellulose acetate solution for use as an outer layer cellulose acylate dope.

Matting agent solution

| | |
|---|---|
| Silica particles with an average particle size of 20 nm | 2 parts by mass |
| (AEROSIL R972, manufactured by Nippon Aerosil Co., Ltd.) | |
| Methylene chloride (first solvent) | 76 parts by mass |
| Methanol (second solvent) | 11 parts by mass |
| The core layer cellulose aeylate dope | 1 part by mass |

<Film Formation for Cellulose Acylate Film 1>

The core layer cellulose acylate dope and the outer layer cellulose acylate dope were filtered through a filter paper having an average pore diameter of 34 μm and a sintered metal filter having an average pore diameter of 10 μm, and then all the three layers of the core layer cellulose acylate dope and the outer layer cellulose acylate dopes arranged on both sides thereof were simultaneously cast on a metal band at 20° C. from a casting port (band caster).

After casting, the formed film was peeled from the metal band in a state where the solvent content was approximately 20% by mass, and the both ends of the film in the width direction were fixed with a tenter clip and dried while stretching the film at a stretch ratio of 1.1 times in the transverse direction. Thereafter, by transporting the film between rolls of a heat treatment device, the film was further dried and rolled to manufacture a long cellulose acylate film 1 having a thickness of 20 sm. The core layer of the film had a thickness of 16 μm, and the outer layers arranged on both sides of the core layer each had a thickness of 2 μm. The in-plane retardation of the obtained cellulose acylate film 1 was 0 nm.

[Manufacture of Photo-Alignment Film P-4]

The coating liquid for forming a photo-alignment film P-4 having the following composition was continuously applied onto the cellulose acylate film 1 with a wire bar of #2.4.

The cellulose acylate film 1 on which the coating film had been formed was dried with hot air at 140° C. for 120 seconds, and subsequently irradiated with polarized ultraviolet rays at 10 mJ/cm² (measurement wavelength of 315 nm using a ultra-high pressure mercury lamp) through a wire grid polarizer (ProFlux PPL02, manufactured by Moxtek, Inc.) to form a photo-alignment film P-4.

(Coating Liquid for Forming Photo-Alignment Film P-4)

| | |
|---|---|
| The following polymer PA-1 | 100.00 parts by mass |
| Isopropyl alcohol | 16.50 parts by mass |
| Butyl acetate | 1,072.00 parts by mass |
| Methyl ethyl ketone | 268.00 parts by mass |

Polymer PA-1

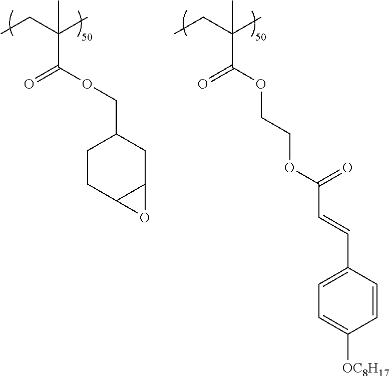

[Formation of Optically Anisotropic Layer]

The following composition A-1 was applied onto the photo-alignment film P-4 using a bar coater. The coating film formed on the photo-alignment film P-4 was heated to 145° C. with hot air and then cooled to 70° C., and the coating film was irradiated with ultraviolet rays at 100 mJ/cm² at a wavelength of 365 nm in a nitrogen atmosphere using a high-pressure mercury lamp and subsequently irradiated with ultraviolet rays at 500 mJ/cm² under heating to 120° C. to fix the alignment of the liquid crystal compound, thereby manufacturing an optical film including an optically anisotropic layer (positive A-plate). The thickness of the optically anisotropic layer is shown in Table 3 below.

(Polymerizable Composition)

| | |
|---|---|
| The polymerizabie liquid crystal compound L-1 | 32.00 parts by mass |
| The polymerizable liquid crystal compound L-2 | 48.00 parts by mass |
| Compounds described in Table 5 below | 20.00 parts by mass |
| The polymerization initiator PI-1 | 0.50 parts by mass |
| The leveling agent T-1 | 0.20 parts by mass |
| Cyclopentanone | 235.00 parts by mass |

[Manufacture of Polarizing Plate]

<Formation of Positive C-plate C-1>

A film C-1 having a positive C-plate C-1 on a temporary support for formation was manufactured by the same method as for the positive C-plate described in paragraph [0124] of JP2015-200861A. It should be noted that the thickness of the positive C-plate was controlled so that Rth(550) was −69 nm.

<Formation of Polarizing Plate>

A surface of a TD80UL (manufactured by FUJIFILM Corporation) which is a support was subjected to an alkali saponification treatment. Specifically, the support was immersed in a 1.5 N aqueous sodium hydroxide solution at 55° C. for 2 minutes, and the extracted support was washed in a water-washing bathtub at room temperature and neutralized with 0.1 N sulfuric acid at 30° C. Thereafter, the obtained support was washed in a water-washing bathtub at room temperature and further dried with a hot air at 100° C.

Subsequently, a roll-shaped polyvinyl alcohol film having a thickness of 80 μm was continuously stretched five times in an iodine aqueous solution, and the stretched film was dried to obtain a polarizer having a thickness of 20 μm.

The obtained polarizer was bonded to a support (TD80UL) which had been subjected to an alkali saponification treatment were bonded to each other to obtain a polarizing plate 0 in which the polarizer was exposed on one side.

Next, the polarizer of the polarizing plate 0 and the coating surface of the positive A-plate were bonded to each other using a pressure sensitive adhesive (SK-2057, manufactured by Soken Chemical Co., Ltd.) so that the absorption axis of the polarizer and the slow axis of the optically anisotropic layer (positive A-plate) manufactured in Examples 31 to 33 were orthogonal to each other.

Then, only the positive A-plate was transferred onto the polarizing plate by peeling the polarizing plate from the film or the glass plate.

Subsequently, the coating surface of the positive C-plate C-1 in the film C-1 was bonded onto a surface of the transferred positive A-plate using a pressure sensitive adhesive (SK-2057, manufactured by Soken Chemical Co., Ltd.), and the support of the film C-1 was peeled to transfer only the positive C-plate C-1 onto the positive A-plate A, thereby manufacturing polarizing plates 1 to 3.

[Manufacture of Liquid Crystal Display Device]

A polarizing plate on the visible side was peeled from a liquid crystal cell of iPad (registered trademark, manufactured by Apple) and used as an IPS-mode liquid crystal cell. Each of the polarizing plates 1 to 3 manufactured above instead of the peeled polarizing plate was as bonded onto the liquid crystal cell to manufacture a liquid crystal display device. At this time, the bonding was performed such that the absorption axis of the polarizing plate and the optical axis of the liquid crystal layer in the liquid crystal cell were perpendicular to each other as observed from a direction vertical to the substrate surface of the liquid crystal cell at the time of a voltage being off.

[Evaluation 3]

For the measurement of the display performance, a commercially available measuring apparatus for a liquid crystal viewing angle and chromaticity characteristics, Ezcontrast (manufactured by ELDIM), was used, and for the backlight, a commercially available liquid crystal display device iPad (registered trademark, manufactured by Apple) was used. The liquid crystal cell to which the polarizing plate had been bonded was placed so that the optically anisotropic layer was on the side opposite to the backlight side, and measured.

<Measurement of Optical Characteristics>

The light incidence angle dependence of Re was measured at wavelengths of 450 nm and 550 nm, using AxoScan OPMF-1 (manufactured by Opto Science, Inc.). The results are shown in Table 5 below.

<Contrast>

In order to set a standard for evaluation, a polarizing plate 0 in which the positive A-plate and the positive C-plate had not been bonded to each other was directly bonded to a liquid crystal display device.

A luminance (Yw) in the direction vertical to a panel in a white display and a luminance (Yb) in the direction vertical to a panel in a black display were measured using a commercially available measuring apparatus for a liquid crystal viewing angle and chromaticity characteristics, Ezcontrast (manufactured by ELDIM), and a contrast ratio (Yw/Yb) in the direction vertical to the panel was calculated and taken as a front contrast, and evaluated according to the following standard. The results are shown in Table 3 below.

A: The front contrast is 95% or more with respect to the polarizing plate 0.

B: The front contrast is 85% or more and less than 95% with respect to polarizing plate 0.

C: The front contrast is 75% or more and less than 85% with respect to polarizing plate 0.

D: The front contrast is less than 75% with respect to polarizing plate 0.

<Moisture-Heat Resistance>

Glass was further bonded onto the polarizing plate bonded to the liquid crystal display device, using a pressure sensitive adhesive, and after being left at 500 hours at 85° C., it was compared with the same sample which had not been exposed to a high temperature to evaluate a tint change during black display. The results are shown in Table 5 below.

A: A tint change is not visually recognized for a sample which has not been exposed to a high temperature.

B: A tint change within an acceptable range is perceived for a sample which has not been exposed to a high temperature.

C: A tint change is significant and not acceptable for a sample which has not been exposed to a high temperature.

<Surface Condition>

In a case where a surface condition of the manufactured optical film was visually confirmed with a polarization microscope, no bright spots or streaks were found, and thus, the film was evaluated as “A” in Table 5 below.

<X-Ray Diffraction Measurement>

X-ray diffraction measurement was performed under the following conditions and the diffracted light due to the orderliness of the smectic phase was confirmed. The results are shown in Table 5 below. With regard to the “Layer structure” in Table 5 below, a case where the diffracted light can be confirmed is denoted as “Yes”.

(Apparatus and Conditions)

X-ray diffractometer ATXG, Cu source (50 kV·300 mA), 0.45 solar slit

TABLE 5

| | | Optically anisotropic layer | | | | | | | | | | |
|---|---|---|---|---|---|---|---|---|---|---|---|---|
| | | Polymerizable | | | | | | Evaluation | | | | |
| | Phase | liquid | | Phase | Thick- | | Photo- | | Re450/ | | Moist- | |
| | difference | crystal | Com- | transition | ness | Layer | alignment | Re550 | Re550 | | heat | Surface |
| Table 5 | plate | compound | pound | temperature | (μm) | structure | film | (nm) | (nm) | Constrast | resistance | condition |
| Example | Phase | L-1 | — | Cr 136 SA | 2.8 | Present | P-4 | 144 | 0.86 | A | A | A |
| 31 | difference | | | 198 N 250 or | | | | | | | | |
| | plate 1 | | | more Is | | | | | | | | |
| | | L-2 | — | Cr 143 | | | | | | | | |
| | | | | N 208 Is | | | | | | | | |
| | | — | 1-3 | Cr 135-140 | | | | | | | | |
| | | | | N 247 Is | | | | | | | | |
| Example | Phase | L-1 | — | Cr 136 SA | 2.8 | Present | P-4 | 144 | 0.86 | A | A | A |
| 32 | difference | | | 198 N 250 or | | | | | | | | |
| | plate 2 | | | more Is | | | | | | | | |
| | | L-2 | — | Cr 143 | | | | | | | | |
| | | | | N 208 Is | | | | | | | | |
| | | — | 1-6 | Cr 117 | | | | | | | | |
| | | | | SA 162 | | | | | | | | |
| | | | | N 255 Is | | | | | | | | |
| Example | Phase | L-1 | — | Cr 136 SA | 2.8 | Present | P-4 | 144 | 0.86 | A | A | A |
| 33 | difference | | | 198 N 250 or | | | | | | | | |
| | plate 3 | | | more Is | | | | | | | | |
| | | L-2 | — | Cr 143 N | | | | | | | | |
| | | | | 208 Is | | | | | | | | |
| | | — | 1-11 | Cr 82 SA | | | | | | | | |
| | | | | 197 N 255 Is | | | | | | | | |

From the results shown in Table 5 above, it was found that even in a case where optical anisotropy was formed using the compound of the embodiment of the present invention, the film contrast of the optical film was excellent, and the moisture-heat resistance and the surface condition were also good (Examples 31 to 33).

### EXPLANATION OF REFERENCES

10: optical film
12: optically anisotropic layer
14: alignment film
16: support
18: hard coat layer

What is claimed is:

1. A compound represented by Formula (1), 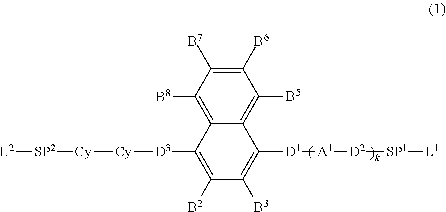
in Formula (1),
A¹ represents an aromatic ring which may have a substituent or an alicyclic ring which may have a substituent,
Cy represents a 1,4-cyclohexylene group which may have a substituent, and the two Cy's may be the same as or different from each other,
D¹, D², and D³ each independently represent a single bond, or a divalent linking group consisting of —O—, —CO—, —S—, —C(═S)—, —CR¹R²—, —CR¹═CR²—, —NR¹—, or a combination of two or more thereof, and R¹ and R² each independently represent a hydrogen atom, a fluorine atom, or an alkyl group having 1 to 4 carbon atoms,
SP¹ and SP² each independently represent a single bond, a linear or branched alkylene group having 1 to 12 carbon atoms, or a divalent linking group obtained by each independently substituting one or more of —CH₂-'s constituting a linear or branched alkylene group having 1 to 12 carbon atoms with —O—, —CO—, —S—, —C(═S)—, —CR¹R²—, —CR¹═CR²—, or —NR¹—, and R¹ and R² each independently represent a hydrogen atom, a fluorine atom, or an alkyl group having 1 to 4 carbon atoms,
k represents an integer of 1 to 3, and in a case where k is 2 or 3, a plurality of A¹'s and D²'s which are present in the formula may be the same as or different from each other,
L¹ and L² each independently represent a monovalent organic group, and at least one of L¹ or L² represents a polymerizable group, and
B², B³, B⁵, B⁶, B⁷, and B⁸ each independently represent a hydrogen atom or a substituent, provided that in a case where at least one of B², B³, B⁶, or B⁷ represents a substituent, the substituent does not include a ring structure.

2. A compound represented by Formula (2),
wherein the compound is other than the compound according to claim 1, 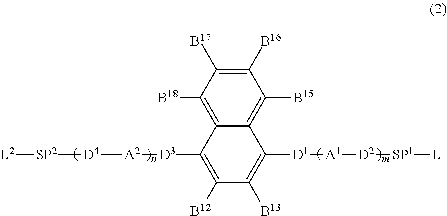
in Formula (2),
A¹ and A² each independently represent an aromatic ring which may have a substituent or an alicyclic ring which may have a substituent,
D¹, D², D³, and D⁴ each independently represent a single bond, or a divalent linking group consisting of —O—, —CO—, —S—, —C(═S)—, —CR¹R²—, —CR¹═CR²—, —NR¹—, or a combination of two or more thereof and R¹ and R² each independently represent a hydrogen atom, a fluorine atom, or an alkyl group having 1 to 4 carbon atoms,
SP¹ and SP² each independently represent a single bond, a linear or branched alkylene group having 1 to 12 carbon atoms, or a divalent linking group obtained by each independently substituting one or more of —CH₂-'s constituting a linear or branched alkylene group having 1 to 12 carbon atoms with —O—, —CO—, —S—, —C(═S)—, —CR¹R²—, —CR¹═CR²—, or —NR¹—, and R¹ and R² each independently represent a hydrogen atom, a fluorine atom, or an alkyl group having 1 to 4 carbon atoms,
m and n each independently represent an integer of 1 to 3, and satisfy m+n=an integer of 3 to 6, in a case where m is 2 or 3, A¹'s and D²'s which are present in the formula may be the same as or different from each other, and in a case where n is 2 or 3, A²'s and D⁴'s which are present in the formula may be the same as or different from each other,
L¹ and L² each independently represent a monovalent organic group, and at least one of L¹ or L² represents a polymerizable group, and
B¹², B¹³, B¹⁵, B¹⁶, B¹⁷, and B¹⁸ each independently represent a hydrogen atom or a substituent, provided that in a case where at least one of B¹², B¹³, B¹⁶, or B¹⁷ represents a substituent, the substituent does not include a ring structure, and in a case where at least one of B¹² or B¹³ represents a substituent, the substituent does not include —CHO.

3. The compound according to claim 1,
wherein L¹ or L² in Formula (1) each represent a polymerizable group.

4. The compound according to claim 1,
wherein k in Formula (1) represents an integer of 2 or 3.

5. The compound according to claim 1,
wherein at least one of B², B³, B⁵, B⁶, B⁷, or B⁸ in Formula (1) represents a substituent.

6. The compound according to claim 1,
wherein m and n in Formula (2) each represent an integer of 2 or 3.

7. The compound according to claim 2,
wherein at least one of B¹², B¹³, B¹⁵, B¹⁶, B¹⁷, or B¹⁸ in Formula (2) represents a substituent.

8. The compound according to claim 1,
wherein at least one of B⁵ or B⁸ in Formula (1) represents a substituent.

9. The compound according to claim 2,
wherein at least one of B¹ or B¹⁸ in Formula (2) represents a substituent.

10. The compound according to claim 8,
wherein at least one of B², B³, B⁶, or B⁷ in Formula (1) represents a hydrogen atom.

11. The compound according to claim 9,
wherein at least one of B¹², B¹³, B¹⁶, or B¹⁷ in Formula (2) represents a hydrogen atom.

12. The compound according to claim 1,
wherein at least one of B², B³, B⁶, or B⁷ in Formula (1) represents a substituent.

13. The compound according to claim 2,
wherein at least one of B¹², B¹³, B¹⁶, or B¹⁷ in Formula (2) represents a substituent.

14. The compound according to claim 1,
wherein at least one of B² or B³ in Formula (1) represents a substituent.

15. The compound according to claim 2,
wherein at least one of B¹² or B¹³ in Formula (2) represents a substituent.

16. The compound according to claim 14,
wherein at least one of B⁵, B⁶, B⁷, or B⁸ in Formula (1) represents a hydrogen atom.

17. The compound according to claim 15,
wherein at least one of B¹⁵, B¹⁶, B¹⁷, or B¹⁸ in Formula (2) represents a hydrogen atom.

18. The compound according to claim 1,
wherein at least one of B⁶ or B⁷ in Formula (1) represents a substituent.

19. The compound according to claim 2,
wherein at least one of B¹⁶ or B¹⁷ in Formula (2) represents a substituent.

20. The compound according to claim 18,
wherein at least one of B², B³, B⁵, or B⁸ in Formula (1) represents a hydrogen atom.

21. The compound according to claim 19,
wherein at least one of B¹², B¹³, B¹⁵, or B¹⁸ in Formula (2) represents a hydrogen atom.

22. The compound according to claim 1,
wherein at least one of B², B³, B⁵, B⁶, B⁷, or B^{B} in Formula (1) represents a substituent, and the substituent represents an alkyl group, an alkoxy group, an alkylcarbonyl group, an alkoxycarbonyl group, an alkylcarbonyloxy group, an alkylamino group, a dialkylamino group, an alkylamide group, an alkenyl group, an alkynyl group, halogen, a cyano group, a nitro group, an alkylthiol group, an N-alkylcarbamate group, an aryl group, an aryloxy group, an arylcarbonyl group, an arylcarbonyloxy group, an arylamino group, an arylamide group, an arylthiol group, an N-arylcarbamate group, a cycloalkyl group, a cycloalkyloxy group, a cycloalkylcarbonyl group, a cycloalkylcarbonyloxy group, a cycloalkylamino group, a cycloalkylamide group, a cycloalkylthiol group, an N-cycloalkylcarbamate group, a sulfonic acid ester group, or a monovalent organic group obtained by each independently substituting one or more of —CH₂-'s constituting an alkyl group with —O— or —CO—.

23. The compound according to claim 2,
wherein at least one of B¹², B¹³, B¹⁵, B¹⁶, B¹⁷, or B¹⁸ in Formula (2) represents a substituent, and the substituent represents an alkyl group, an alkoxy group, an alkylcarbonyl group, an alkoxycarbonyl group, an alkylcarbonyloxy group, an alkylamino group, a dialkylamino group, an alkylamide group, an alkenyl group, an alkynyl group, halogen, a cyano group, a nitro group, an alkylthiol group, an N-alkylcarbamate group, an aryl group, an aryloxy group, an arylcarbonyl group, an arylcarbonyloxy group, an arylamino group, an arylamide group, an arylthiol group, an N-arylcarbamate group, a cycloalkyl group, a cycloalkyloxy group, a cycloalkylcarbonyl group, a cycloalkylcarbonyloxy group, a cycloalkylamino group, a cycloalkylamide group, a cycloalkylthiol group, an N-cycloalkylcarbamate group, a sulfonic acid ester group, or a monovalent organic group obtained by each independently substituting one or more of —CH₂-'s constituting an alkyl group with —O— or —CO—.

24. A polymerizable composition comprising the compound according to claim 1.

25. The polymerizable composition according to claim 24, further comprising a polymerizable liquid crystal compound different from the compound.

26. The polymerizable composition according to claim 24, further comprising a polymerization initiator.

27. A cured product obtained by curing the polymerizable composition according to claim 24.

28. An optical film comprising the cured product according to claim 27.

29. A polarizing plate comprising:
the optical film according to claim 28; and
a polarizer.

30. An image display device comprising the optical film according to claim 28.
